(19) United States
(12) Patent Application Publication
(10) Pub. No.: US 2004/0078015 A1
(43) Pub. Date: Apr. 22, 2004

(54) Extruded super absorbent web

(76) Inventors: Marcelo S. Copat, Terre Haute, IN (US); Niloufar Faridi, Melville, NY (US); Laurence Tim Pearson, West Terre Haute, IN (US); Darrel Loel Wilhoit, Washougal, WA (US); Bakhitar Alam Shah, Terre Haute, IN (US)

Correspondence Address: HUNTON & WILLIAMS LLP, INTELLECTUAL PROPERTY DEPARTMENT, 1900 K STREET, N.W., SUITE 1200, WASHINGTON, DC, 20006-1109, US

(21) Appl. No.: 10/462,752

(22) Filed: Jun. 17, 2003

Related U.S. Application Data
(60) Provisional application No. 60/388,746, filed on Jun. 17, 2002.

(51) Int. Cl. A61F 13/15; A61F 13/20

(52) U.S. Cl. 604/370

(57) ABSTRACT

An absorbent article including at least one of a topsheet, an absorbent core, an optional distribution layer and a backsheet, at least one of which comprises at least one layer of an extruded superabsorbent web is disclosed. The superabsorbent webs can be made by heating and mixing blends of thermoplastic resins and absorbent polymers in a continuous process, and then preferably extruding the web. The extruded superabsorbent web can be flat or formed, stretched or unstretched, and coextruded or laminated with or to other materials.

### BACKGROUND OF THE INVENTION

1. Technical Field of the Invention

The present invention generally relates to absorbent articles comprising absorbent materials, and to methods for forming superabsorbent materials and products produced therefrom. More specifically, the invention relates to extruded superabsorbent materials, and to the methods of making the same.

2. Description of Related Art

One objective of developments in the absorbent article field is to provide both a high level of protection and a high comfort level to the wearer. Another objective is to reduce the total cost of the absorbent article.

One mechanism for providing consumer comfort benefits in absorbent articles is by the provision of breathable products. Breathability has typically concentrated on the incorporation of so called “breathable backsheets” in the absorbent articles. Commonly utilized breathable backsheets are microporous films and apertured formed films having directional fluid transfer as disclosed in for example, U.S. Pat. No. 4,591,523. Both of these types of breathable backsheets allow the evaporation of a portion of the fluid stored in the absorbent core and increase the circulation of air within the absorbent article. The air circulation is particularly beneficial as it reduces the sticky feeling experienced by many wearers during use, commonly associated with the presence of an apertured formed film or film like topsheet.

A well known problem associated with the use of breathable backsheets is that of liquid passage onto wearer's garments. Attempts to solve the problem have mainly resided in the use of multiple layer backsheets such as those illustrated in U.S. Pat. No. 4,591,523. Similarly European patent application Nos. EP 0 710 471 and EP 0 710 472 disclose breathable backsheets comprising layers of a gas permeable fibrous fabric and layers of apertured formed films having directional fluid transport. Such backsheets permit liquid passage when pressure is applied to the absorbent article (or the “pad”). The amount of pressure required to cause liquid passage is inversely proportional to the diameter of the capillaries. Because the passage of gasses is also proportional to the diameter of the capillaries, improved leakage protection reduces the breathability of the backsheet.

European patent application Nos. EP 0 934 735 and EP 0 934 736 improve upon EP 0 710 471 and EP 0 710 472 by incorporating in a backsheet of an absorbent article angled apertured formed film which has improved fluid management characteristics. Such films are also described in commonly assigned U.S. Pat. Nos. 5,562,932, 5,591,510, 5,718,928, and 5,897,543.

None of the above proposed developments has been able to provide a fully satisfactory solution to the problem of a breathable backsheet that allows minimum, if any, liquid passage under substantially all possible conditions. Therefore, the prior art is limited by the competing requirements of breathability and liquid passage suppression.

Another mechanism for providing consumer comfort benefits in absorbent articles (or “articles”) is by providing absorbent cores that quickly acquire bodily fluids transmitted from the body contacting surface of the pad, keep the fluids from returning to the body contacting surface when pressure is applied to the pad, and distribute the fluid evenly within the absorbent core so that core utilization is maximized. Core utility maximization enables reduction of core size that results in smaller, more comfortable pads. A problem encountered with absorbent articles that are intended to repeatedly receive and absorb body liquid, or fluid, discharged by a user is that the rate at which the liquid is able to penetrate into the article decreases considerably with each new wetting occasion, or insult.

The reason why the body liquid penetration rate decreases with repeated wetting of the article is because the absorbent body of the article becomes saturated with body liquid temporarily within a limited area around the area on the article surface in which the body liquid first impinges, the so-called primary wetting (or insult) area. The absorbent cores are normally comprised of one or more layers of hydrophilic fibers, for instance cellulose fluff pulp, and often also include a powerful absorbing hydrocolloidal material, so-called superabsorbents. Liquid is transported relatively slowly through such materials, since transportation of the liquid is mainly caused by the capillary forces acting in the cavities located between fibers and particles in the absorbent body of the article. Liquid is transported within the hydrocolloidal materials by diffusion, which is a still slower process than the process generated by the capillary forces. The liquid will therefore remain in the primary wetting area of the article for a relatively long period of time and will then gradually be transported out to surrounding parts of the absorbent body.

Superabsorbent polymers typically are synthetic cross-linked polymeric materials that are capable of absorbing many times their own weight in water and other liquids. Because superabsorbent polymers are significantly cross-linked, it is virtually impossible to dissolve (or solvate) them into solution. Accordingly, superabsorbent polymers are most commonly used as powders or granules. The use of superabsorbent polymers in these physical forms presents product design problems as well as health risks. For example, the powdered material has a natural tendency to bunch up or agglomerate within the supporting matrix of the absorbent product. This results in uneven absorptive capacity in the product. Similarly, the fine particulates have a tendency to “dust-off” the supporting matrix resulting in loss of the superabsorbent polymer material altogether.

Powdered superabsorbent polymers also pose health risks both to end users and to those involved in the manufacturing process. The finely powdered SAP can become airborne where it can be inhaled by workers or end users. Once inhaled, the SAP absorbs liquid within the respiratory passages swelling to many times its original size. This can result in blocked air passages and potentially traumatic health complications.

A conventional approach has been to simply disperse the powdered SAP material in a solid matrix material (e.g. wood pulp, cotton batting, etc.) and fix it in place mechanically as by embossing. EP 0 212 618 B1 describes diaper constructions wherein polymerizates having a specific grain size distribution are distributed in a cellulose fiber layer. However, such a construction is insufficiently stable with respect to the distribution of the superabsorbent polymer. Specifically; the distribution of the SAP may be altered undesirably during transportation, resulting in non-uniform absorption, e.g., in a diaper.

EP 0 255 654 suggests the fabrication of dry formed sheets incorporating cellulose fibers and SAPs. The two materials are suspended in an air stream, fed to a head for dry-forming sheets of paper, laid down on a web, and bound by calendaring and embossing.

To eliminate added processing steps, U.S. Pat. No. 4,826,880 suggests forming hydrates of the SAPs. Such hydrates have reduced tendency to dust off a product and can be used in routine coating processes to coat conventional substrates such as cloth, nonwovens of various fibers, and vinyl films. These hydrates have reduced absorptive properties.

Other approaches glue or otherwise adhere the particulate SAP material to a fibrous material, which then is mechanically immobilized in the substrate. WO 90/11181 discloses bicomponent fiber products in which fibers are coated with a liquid binder material. While the binder material is still wet, the particulate SAP is applied resulting in a comprehensive and uniform coating of the matrix fibers. The fibers then are fixed in a fabric or similar substrate by embossing or some such manner. Still other approaches seek to affix the particulate SAP material to a matrix chemically.

EP 0 425 269 A1 discloses melt-spinnable fibers from thermoplastic materials containing SAP, whereby SAP materials have been blended with thermoplastic materials for melt extrusion. Cellulose acetate is disclosed among the materials contemplated for these fibers. EP 0 425 269 A1 teaches that the upper limit of SAP in melt extrusion is 30% by weight. Beyond that point desired qualities of the product are lost. EP 0 425 269 A1 also teaches a method of fixing powdered SAPs to thermoplastic, water-insoluble fibers. Binding the SAP to the fiber is effected in such a way that a slightly surface-melted fiber is contacted with the powdered superabsorbent polymer. The fibers themselves are fixed among one another in the same manner. A disadvantage of this process is that the absorptive capacity of the powdered superabsorbent polymer is not utilized to its full extent. Part of the SAP is covered by the thermoplastic and thus not reached by water or aqueous solutions.

EP 0 547 474 A1 describes superabsorbent materials in the form of sheets or fibers made from high melting polymers and having superabsorbent polymer material dispersed uniformly throughout and immobilized in a hardened polymer matrix. The materials are capable of incorporating large amounts of superabsorbent polymer and so demonstrate substantially improved absorbency and retention properties. The absorbent material is fabricated by forming a liquid mixture of the matrix material and a suitable solvent. Such a liquid mixture is known generally within the art as a dope. Often the liquid mixture is a solution wherein the matrix material is completely solvated by a solvent. The dope is supplemented with particulate or powdered superabsorbent polymer. Because of its substantially cross-linked character, the SAP is not solvated but remains as a suspension in the dope. The dope is extruded or cast to form sheets, films or fibers of matrix material having the SAP particulates embedded throughout. The resulting absorbent material is a matrix material within which is securely immobilized a SAP material. The patent shows that in the range of 25-50% SAP content the absorbency (g/g) is in the range of 9-16.

JP Application No. 75-85462 describes a method of producing superabsorbent sheets made of a starch/graft polymer integrated in a water-soluble, film-forming polymer. This document discloses a material serving as base material as an indispensable third component of the sheet. The superabsorbent polymer is fixed on the base material together with the soluble, film-forming polymer.

None of the above proposed developments has been capable of providing a fully satisfactory solution to the problem of liquid absorption and retention in an article that is also comfortable to wear and cost effective to manufacture such that inexpensive articles can be offered to the market.

The description herein of disadvantages and inferior properties attained with known products, processes and apparatus is in no way intended to limit the scope of the invention. Indeed, certain aspects of the invention may include one or more known materials, processes, and apparatus without suffering from the disadvantages and inferior properties so described. All United States patents mentioned in this description are incorporated by reference herein in their entirety.

### SUMMARY OF THE INVENTION

The present invention relates to absorbent articles, such as baby diapers, adult incontinent articles and in particular to sanitary napkins or panty liners. The articles usually include an absorbent core disposed at least partially between a liquid pervious topsheet and a liquid impervious backsheet. Optionally, the articles may include an acquisition/distribution layer (ADL) disposed between the topsheet and said backsheet. At least one of the layers included in at least one of the topsheet, the absorbent core, the ADL, or the backsheet, comprises an extruded superabsorbent web. The invention also is directed to the extruded superabsorbent web and to the method of making an extruded superabsorbent web.

Webs of the invention also are useful in other applications such as in absorbent packaging articles, non-absorbent articles, infection control products, household cleaning products, and industrial cleaning products, sweat bands, and the like.

The instant invention is based in part upon the discovery that large amounts of SAP can be dry blended and extruded in combination with thermoplastic polymers to form superabsorbent webs, and that upon stretching of such webs the absorptive capacity of the superabsorbent web is sufficiently high to be practical. The methods and apparatus utilized to realize the invention are simple and productive, thereby enabling the creation of useful and less expensive superabsorbent materials.

Various embodiments of the invention relate to absorbent articles, such as disposable absorbent articles of a layered construction, such as baby diapers, adult incontinent articles, bandages and underarm sweat pads, and in particular sanitary napkins or panty liners. Other embodiments relate to absorbent articles such as oil sorbent products, sanitary wipes, meat trays, and the like. Typically, such articles comprise a liquid pervious topsheet, an absorbent core (or “core”), and a backsheet. In hygiene absorbent articles the topsheet contacts the wearer and the backsheet usually is breathable and forms the garment facing (or contacting) surface of the article. The absorbent core typically is disposed at least partially between the topsheet and the backsheet. The absorbent core includes at least one absorbent material, such as a hydrogel, a superabsorbent, or a hydrocolloid material, in combination with suitable carriers. In accordance with a feature of the invention, at least one of the layers included in at least one of the topsheet, the absorbent core or the backsheet of an embodiment of the invention comprises an extruded superabsorbent web.

An embodiment of the present invention also generally relates to extruded superabsorbent webs, and to methods for forming extruded superabsorbent webs and the webs made thereby. More specific embodiments of the invention relate to extruded superabsorbent webs, and to methods of making the same. The extruded superabsorbent webs preferably comprise a blend of at least one thermoplastic resin and at least one superabsorbent polymer. The method of forming the extruded superabsorbent webs preferably comprises blending at least one thermoplastic resin with at least one superabsorbent polymer, melting the thermoplastic resin, mixing the molten thermoplastic resin with the superabsorbent polymer to form a molten blend of superabsorbent polymer and resin, extruding the molten blend through an extrusion die to form a molten sheet, and cooling the molten sheet to form an extruded web. In a preferred embodiment, the extruded web may be wound into a roll. In an additional preferred embodiment, the cooled extruded superabsorbent web is stretched prior to winding to increase the absorption capacity of the web.

In another embodiment of the invention, there is provided an extruded superabsorbent web that comprises at least two layers, a first layer comprising a blend of at least one thermoplastic resin, optional additives, and at least one superabsorbent polymer, and a second layer comprising a blend of at least one thermoplastic resin, optional additives, and at least one superabsorbent polymer present in an amount less than the amount of the at least one superabsorbent polymer present in the first layer. The optional additives can include processing aids and colorants, and they may also include fillers such as calcium carbonate. The at least two layers can be co-extruded or they can be combined subsequent to extrusion. Upon stretching, the web can be rendered breathable, thereby converting the at least two-layer substrate into a multi-functional composite web that functions both as a superabsorbent web and as a breathable backsheet, thereby reducing the complexity of the article, reducing its bulk, improving “hand”, and reducing cost.

In yet another embodiment of the invention, there is provided an extruded superabsorbent web comprising at least two layers, a first layer comprising a blend of at least one thermoplastic resin, optional additives, and at least one superabsorbent polymer, and a second layer comprising a nonwoven web. The optional additives can include processing aids and colorants, and may also include fillers such as calcium carbonate. The at least two layers preferably are combined subsequent to extrusion of the first layer. Upon stretching, the web may be rendered breathable, thereby converting the at least two-layer substrate into a multi-functional composite web that functions both as a superabsorbent web and as a breathable backsheet.

In an additional embodiment of the invention, there is provided an extruded superabsorbent web comprising at least three layers, a first layer comprising a blend of at least one thermoplastic resin, optional additives, and at least one superabsorbent polymer, a second layer comprising a blend of at least one thermoplastic resin, optional additives, and at least one superabsorbent polymer present in an amount less than the amount present in the first layer, and a third layer comprising a nonwoven web. The optional additives can include processing aids and colorants, and may also include fillers such as calcium carbonate. The first and second layers can be co-extruded or can be combined subsequent to extrusion. The third layer preferably is combined with the first and second layers subsequent to extrusion of the first layer. Upon stretching, the web may optionally be rendered breathable, thereby converting the at least three-layer substrate into a multi-functional composite web that functions both as a superabsorbent web and as a breathable backsheet.

The first or second layer, or both film layers of any of the above embodiments can be a three-dimensional formed film, and such formed film can be either apertured or unapertured. Use of formed films expands the surface area available to absorb. Unapertured formed films are most useful in wipe applications and meat tray applications where high absorption capacity is required but where breathability is not necessary. Such composite materials also are useful in medical applications where the primary function of the material is to absorb blood—such as in operating room drapes—without allowing blood to pass through the composite web.

In another embodiment of the present invention, there is provided an extruded superabsorbent web comprising a formed film containing at least the superabsorbent web, and optionally containing at least two layers, a first layer comprising a blend of at least one thermoplastic resin, optional additives, and optionally superabsorbent polymers, and a second layer comprising a blend of at least one thermoplastic resin, optional additives, and at least one superabsorbent polymer present in an amount greater than the amount, if any, present in the first layer. The first and second layers may be co-extruded. An optional third layer comprising a nonwoven web can be combined with the at least two-layer formed web by bonding it to the second layer subsequent to extrusion of the formed web. This composite construction may be useful as a backsheet. The male protrusions of the formed film preferably are oriented towards the absorbent core. In such a construction the purpose of the superabsorbent polymer is to absorb any fluid that may pass through the male protrusions of the formed film and to prevent such fluid from reaching the outer surface of the backsheet.

### BRIEF DESCRIPTION OF THE DRAWINGS

FIG. 1 is a schematic view of a preferred apparatus for carrying out the method of this invention.

FIG. 1A is a schematic view of a preferred embodiment of the method of this invention. FIG. 1A exemplifies a process where the absorbent web is not stretched. The web can be flat or formed.

FIG. 1B is a schematic view of a preferred embodiment of the method of this invention. FIG. 1B exemplifies a process where the SAP polymer is compounded first and subsequently the pre-compounded SAP is extruded to produce an absorbent web.

FIG. 2 is a schematic of another preferred embodiment of the method of this invention. FIG. 2 exemplifies an absorbent web laminated via extrusion lamination or vacuum lamination to a flat or formed molten sheet to produce absorbent webs such as those shown in FIGS. 4A, 6, and 8.

FIG. 3 is a schematic of another preferred embodiment of the method of this invention. FIG. 3 exemplifies a co-extruded absorbent web to produce absorbent webs such as those shown in FIGS. 5-9.

FIG. 4 is a cross sectional view of an extruded superabsorbent web of this invention.

FIG. 4A is a cross sectional view of another extruded superabsorbent web of this invention.

FIG. 5 is a cross sectional view of another extruded superabsorbent web of this invention.

FIG. 6 is a cross sectional view of another extruded superabsorbent web of this invention.

FIG. 7 is a cross sectional view of another extruded superabsorbent web of this invention.

FIG. 8 is a cross sectional view of another extruded superabsorbent web of this invention.

FIG. 9 is a cross sectional view of another extruded superabsorbent web of this invention.

FIG. 9A is a cross sectional view of a formed film made in accordance with the invention, and FIG. 9B is a cross sectional view of a stretched formed film made in accordance with the invention.

FIG. 10 is a cross sectional view of another extruded superabsorbent web of this invention.

FIG. 11 is a cross sectional view of intermeshing gear (IMG) teeth.

FIG. 12 is a cross sectional view of a machine direction orientation IMG roll.

FIG. 13 is a cross sectional view of a transverse machine direction orientation IMG roll.

FIG. 14 is a highly expanded cross sectional view of a stretched monolayer extruded superabsorbent web of the invention.

FIG. 15 is a cross sectional view of a stretched monolayer extruded superabsorbent web of the invention.

FIG. 16 is a highly expanded surface view of a stretched monolayer extruded superabsorbent web of the invention.

FIG. 17 is a graph depicting the additional absorbency obtained by increasing the amount of SAP in the film formulation.

FIG. 18 is a graph depicting the improvement in SAP absorption rate obtained by stretching the unstretched film of example 7 at various process conditions A, B, C and D.

FIG. 19 is a chart depicting the improvement in SAP absorption rate obtained by stretching the unstretched film of example 8 at various process conditions A, B, C and D.

FIG. 20 is an illustration of an apparatus useful in measuring the absorption under load.

FIGS. 21A and 21B are graphs showing the strikethrough times for conventional materials and inventive materials, respectively.

FIG. 22 is a graph showing the strikethrough times for inventive materials in combination with various ADLs.

### DETAILED DESCRIPTION OF THE PREFERRED EMBODIMENTS

Set forth below are definitions of some the terms and phrases used herein.

The expressions “absorbent garment,” “absorbent article” or simply “article” as they are used in throughout this description refer to devices that absorb and contain fluids, body fluids and other body exudates. More specifically, these terms refer to materials used to absorb liquids such as wipes, meat trays, and other absorbent materials, and these terms refer to garments that are placed against or in proximity to the body of a wearer to absorb and contain the various exudates discharged from the body. A non-exhaustive list of examples of absorbent garments includes diapers, diaper covers, disposable diapers, training pants, absorbent underpants, feminine hygiene products and adult incontinence products. Such garments may be intended to be discarded or partially discarded after a single use (“disposable” garments). Such garments may comprise essentially a single inseparable structure (“unitary” garments), or they may comprise replaceable inserts or other interchangeable parts.

The present invention may be used with all of the foregoing classes of absorbent garments, without limitation, whether disposable or otherwise. The invention will be understood to encompass, without limitation, all classes and types of absorbent garments, including those described herein. Preferably, the absorbent composite is thin in order to improve the comfort and appearance of a garment.

Throughout this description, the term “disposed” and the expressions “disposed on,” “disposing on,” “disposed in,” “disposed between” and variations thereof (e.g., a description of the article being “disposed” is interposed between the words “disposed” and “on”) are intended to mean that one element can be integral with another element, or that one element can be a separate structure bonded to or placed with or placed near another element. Thus, a component that is “disposed on” an element of the absorbent garment can be formed or applied directly or indirectly to a surface of the element, formed or applied between layers of a multiple layer element, formed or applied to a substrate that is placed with or near the element, formed or applied within a layer of the element or another substrate, or other variations or combinations thereof.

Throughout this description, the expressions “topsheet” and “backsheet” denote the relationship of these materials or layers with respect to the absorbent core. It is understood that additional layers may be present between the absorbent core and the topsheet and backsheet, and that additional layers and other materials may be present on the side opposite the absorbent core from either the topsheet or the backsheet.

As used herein, the expression “Absorbency” means the amount of a given liquid that is absorbed by a material at a given length of time compared to the weight of the dry material and expressed as “X g/g @Y min” where X is:

(weight of liquid absorbed)/weight of dry sample material

where X is measured after the material is submerged in abundant liquid for Y minutes. The absorbency is determined in accordance with the procedure set forth in the Test Methods section below.

As used herein, the phrases “areas immediately surrounding the region of maximum fluid discharge” or “insult point” denote a surface area surrounding the region of maximum fluid (i.e., liquid) and/or solid waste discharge and extending approximately 1 inch in all directions from that region. Throughout this description, “periphery”, “peripheral areas” or “areas peripheral to” denote the surface area other than the area of maximum fluid discharge and the areas immediately surrounding it.

As used herein, the term “barrier” means a film, laminate or other fabric that is substantially impermeable to the transmission of liquids and that resists a hydrohead of at least 50 mbar water. Hydrohead as used herein refers to a measure of the liquid barrier properties of a fabric. However, it should be noted that barrier fabrics of the present invention can have a hydrohead value greater than 80 mbar, 150 mbar or even 300 mbar water.

As used herein, the term “breathable” refers to a material that is permeable to water vapor having a minimum WVTR of about 300 g/m²/24 hours. The WVTR of a fabric is water vapor transmission rate which, in one aspect, provides an indication of how comfortable a fabric would be to wear. WVTR (water vapor transmission rate) is measured as indicated below and the results are reported in grams/square meter/day. Applications of breathable barriers, however, typically desirably have higher WVTRs, and breathable laminates of the present invention can have WVTRs exceeding about 800 g/m²/day, 1500 g/m²/day, or even exceeding 3000 g/m²/day.

As defined herein, and in the Handbook of Polyethylene: Structure, Properties and Applications, by Andrew Peacock, published by Marcel Dekker, New York, 2000. p. 128, “percent elongation” is defined as the increase in sample length×100 divided by the original sample length.

U.S. Pat. Nos. 4,116,892 and 4,223,059 to Schwartz (the “Schwartz” patents) describe the methodology used to approximate the draw ratio of the incremental stretching process for gears of round tooth design. Gear selection and designs are driven by the desired product characteristics and by the desired process configuration (space limitations, roll diameters, production technique for the rolls and gears etc.) These considerations are discussed in ample detail in the Schwarz patents, the disclosures of each of which are incorporated by reference herein in their entirety.

As used herein, the term “elastic” means a material which, upon application of a biasing force, is stretchable, that is extensible, to a stretched, biased length that is at least 150% of its relaxed unbiased length, and that will retract at least 50% of its elongation upon release of the elongating force. A hypothetical example would be a one (1) inch sample of a material that is elongatable to 1.50 inches and which, upon release of the biasing force, will retract to a length of 1.25 inches or less. This sample is said to have a 25% “set”.

As used herein the term “extensible” means elongatable or stretchable in at least one direction.

As used herein, the expression “Free-Swell Capacity” means the maximum amount of liquid that unrestrained SAP particles will ultimately absorb when exposed to a large amount of a liquid. A given SAP with a given capacity for a given liquid will have a different, somewhat lower capacity for that liquid when the SAP is contained in, for example, an absorbent core of a diaper. Free-Swell Capacity is measured in the same manner and is expressed in the same manner as absorbency with the exception that Y (length of time) is not described.

As used herein, the expression “Absorption Efficiency” means the ratio of the Total Absorption Capacity of a material to the Free-Swell Capacity of the SAP included in such material.

As used herein, the term “garment” means any type of apparel that may be worn. The term garment includes industrial work wear and coveralls, undergarments, pants, shirts, jackets, gloves, socks, and the like.

As used herein, the terms “inelastic” and “non-elastic” refer to any material that does not fall within the definition of “elastic” above.

As used herein, the expression “infection control product” means medically oriented items such as surgical gowns and drapes, face masks, head coverings like bouffant caps, surgical caps and hoods, footwear like shoe coverings, boot covers and slippers, wound dressings, bandages, sterilization wraps, wipers, garments like lab coats, coveralls, aprons and jackets, patient bedding, stretcher and bassinet sheets, and the like.

As used herein, the phrase “non-absorbent articles” means garments, protective covers, and infection control products.

As used herein, the term “permeability” refers to the permeability of a material to a vapor or liquid.

As used herein, the expression “point bonding” means bonding one or more fabrics at a plurality of discrete points. For example, thermal point bonding generally involves passing one or more layers to be bonded between heated rolls such as, for example an engraved pattern roll and an anvil (or smooth calendar) roll. The engraved roll is patterned on its surface in some way so that the entire fabric is not bonded over its entire surface, and the anvil roll usually has a flat or smooth surface. As a result, various patterns for engraved rolls have been developed for functional as well as aesthetic reasons.

As used herein, the phrase “protective cover” means a cover for vehicles such as cars, trucks, boats, airplanes, motorcycles, bicycles, golf carts, etc., covers for equipment often left outdoors like grills, yard and garden equipment (mowers, roto-tillers, etc.) and lawn furniture, as well as floor coverings, table cloths and picnic area covers.

As used herein, the term “set” means the amount of stretch remaining after removal of a biasing force expressed as a percentage of the original length.

As used herein, the expression “ultrasonic bonding” means a process performed, for example, by passing the fabric between a sonic horn and an anvil roll as illustrated in U.S. Pat. No. 4,374,888 to Bornslaeger or in U.S. Pat. No. 5,591,278 to Goodman et al, the disclosures of which are incorporated by reference herein in their entirety.

As used herein, the expression “plasticizing agent” refers to an organic compound which, when added to a high polymer, may increase the ease of processing the high polymer or increase the toughness and flexibility of the high polymer after processing. A plasticizing agent may be able to accomplish all of these. An exemplary plasticizing agent is glycerin.

As used herein, the term “SAP” means a superabsorbent polymer which, when in a substantially dry state, has the ability to spontaneously imbibe more than (20) times its own weight in aqueous fluid, for example, tap water.

As used herein, the expression “SAP speed” means the rate at which the superabsorbent will absorb liquid. The speed of a superabsorbent depends upon many factors including its composition, the quantity of liquid it has already absorbed and the amount of liquid available to be absorbed. In granular form, commercially available superabsorbents take approximately one to three minutes to absorb their free swell capacity of water, when unrestrained and exposed to large amounts of water. When super absorbents are incorporated in absorbent cores with other absorbent materials, such as fluff, they absorb liquid more slowly than this, largely because they are in contact with less liquid, and are restrained. All superabsorbents have a given speed when dry, and, as they absorb liquid, the speed and remaining capacity of the superabsorbent decreases. Ideally, skin will be exposed to a wet diaper surface no more than 10 minutes, i.e. SAP speed should enable absorption of an insult (single discharge of body fluid) in less than 10 minutes.

As used herein, the term “substantially” means that a given property or parameter may vary by about 20% from the stated value.

As used herein, the expression “Total Absorption Capacity” means the maximum amount of liquid that a material will ultimately absorb when exposed to an excessive amount of a liquid. Total Absorption Capacity is measured in the same manner and is expressed in the same manner as Absorption Capacity with the exception that Y (length of time) is not described. The Total Absorption Capacity of unrestrained SAP is its Free-Swell Capacity. Theoretically the Total Absorption Capacity of a material is its Absorption Capacity after an infinite amount of time passed. From a practical point of view the time period can be defined to be such time when the amount of additional liquid absorbed by the material in an hour is less than 1% of the weight of the dry material sample.

Webs contemplated by certain embodiments of the present invention may be made utilizing, for example, polyolefin film processes including blown molding, casting, and cast melt embossing. The preferred process is a cast melt embossed film process. In extrusion processes, the films of the present invention can be formed into a single layer film, or may be one layer or more of a multi-layer film or film composite. The films of the present invention may also be included in laminated structures. As long as a film, multi-layer film, or laminated structure includes one or more layers of an extruded superabsorbent web, such film, multilayer film, or laminated structure will be understood to be contemplated as an embodiment of the present invention.

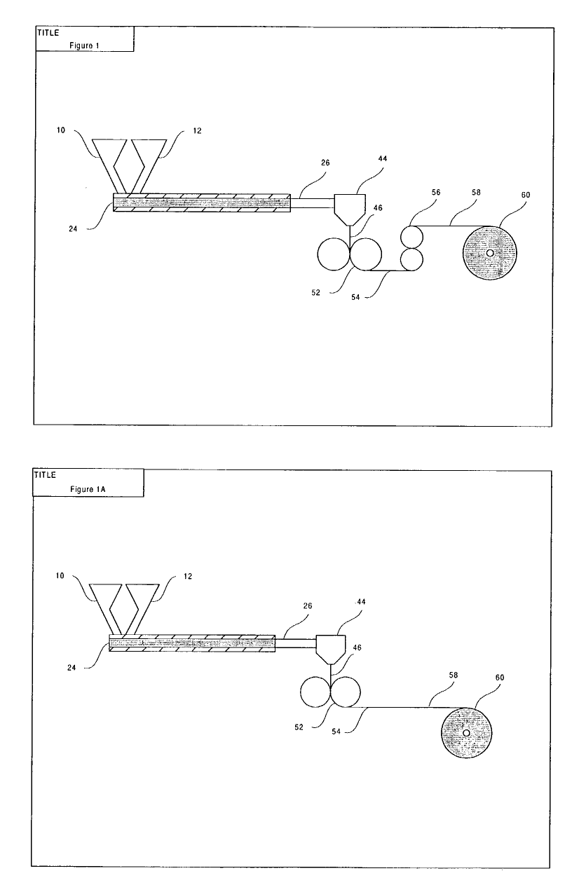

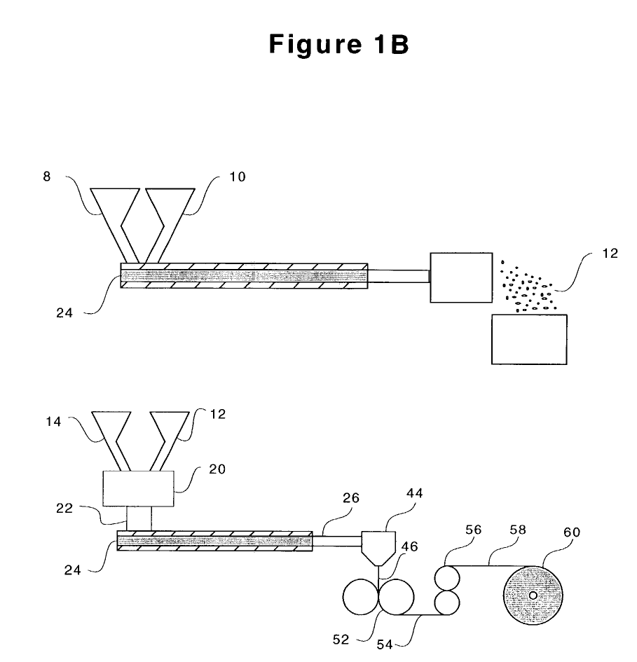

Topsheets—The main function of topsheets is to acquire liquid and to transport it to the inside of the article, albeit an absorbent product, non-absorbent product, infection control product, etc. A co-extruded topsheet with SAP in the inner side, as shown for example in FIG. 9 and as can be made by the process shown in FIG. 1A, would enable the creation of very thin products with enhanced absorption capacity. Importantly, the SAP in the inner layer of the topsheet would help prevent rewet.

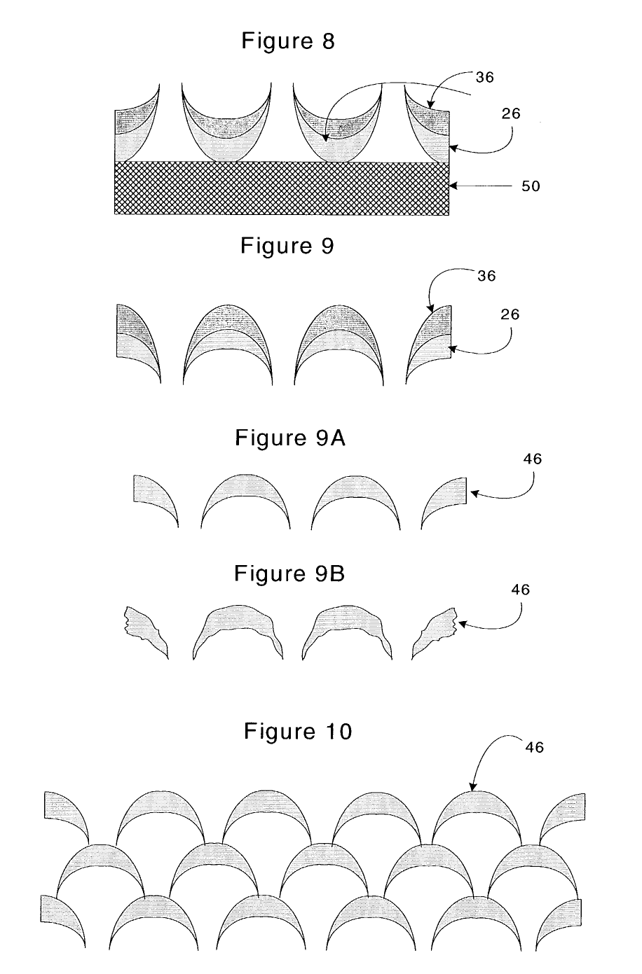

Backsheets—The main function of backsheets is to provide a barrier for articles. Where the backsheet is desired to be breathable, the challenge of providing a breathable barrier can be solved by providing a co-extruded apertured backsheet where the functionality of SAP is to absorb any liquid, albeit small amounts of it, that somehow manages to pass through the male protrusion of the film. Such a film is shown, for example, in FIGS. 7 and 8.

ADL—“Acquisition-Distribution Layer”—The main functions of an ADL are to transport liquid to the inside of the article, while also distributing it over the entire article surface such that most or all of the absorbent material in the article is utilized or exposed to liquid quickly. A co-extruded topsheet with SAP in the inner side, as shown, for example, in FIG. 9 and as can be made by the process shown in FIG. 1A, would enable the creation of very thin products with improved distribution and absorption capacity. Importantly, the SAP in the inner layer of the ADL would help prevent rewet.

### Materials

Any superabsorbent polymer (SAP) can be used in the present invention. Usually, the SAP component is a cellulosic-derived particle, polyacrylic acid based material, and the like. Typically, SAP is produced in granular form, such granules exhibiting a particle size distribution and an average particle size distribution. The average particle size distribution should be related to the un-stretched thickness of the film in such way that the largest particles fit within the thickness of the un-stretched films. Particles produced in larger than adequate sizes may be ground down to the appropriate size, if needed.

Any thermoplastic resin capable of being combined with SAP and extruded can be used in the invention. The thermoplastic resin, or thermoplastic polymer component preferably is any film forming resin including polyethylene and polypropylene, and mixtures thereof, ethylene polar co-monomer polymers, ethylene α-olefin copolymers, ethylene vinyl acetate (EVA), ethylene acrylic acid (EAA), ethylene methacrylic acid (EMA), polystyrene, polyesters, butadiene and other elastomeric thermoplastic resins, and other appropriate thermoplastic polymers and combinations hereof.

It will be understood that, in general, a large number of polyolefins will be useful in the techniques and applications described herein. Also included in the group of polyolefins that are contemplated as embodiments of this invention are metallocene catalyzed polyethylenes, both linear low density and very low density (0.88 to 0.935 g/cm³), high density polyethylene (0.935-0.970 g/cm³), Ziegler-Natta catalyzed linear low density polyethylene, conventional high pressure low density polyethylene (LDPE), and combinations thereof. Various elastomers or other soft polymers may be blended together. Blends include styrene-isoprene-styrene (styrenic block co-polymer), styrene-butadiene-styrene (styrenic block co-polymer), styrene-ethylene/butylene-styrene (styrenic block co-polymer), ethylene-propylene (rubber), propylene homopolymer/ethylene-propylene copolymer impact copolymer mixtures and blends, ethylene-propylene-diene-modified (rubber), ethylene-vinly-acetate, ethylene-methacrylate, ethylene-ethyl-acrylate, and ethylene-butyl-acrylate.

Commonly assigned U.S. Pat. Nos. 5,733,628 and 6,303,208 describe materials used to produce elastomeric films and laminate structures contemplated as part of the invention. The disclosures of these patents are incorporated by reference herein in their entirety.

Other components may also be included in the extruded superabsorbent webs of the invention, such as additives, surfactants, fillers, and the like. These additives, surfactants, fillers, and the like also are useful in materials that do not contain any superabsorbent, but that are coextruded with the superabsorbent webs of the invention. Fillers useful in this invention may include any inorganic or organic material having a low affinity for and a significantly lower elasticity than the film forming thermoplastic component. Preferably the filler, if used, should be a rigid material having a non-smooth hydrophobic surface, or a material that is treated to render its surface hydrophobic. The preferred mean average particle size of suitable fillers preferably is between about 0.5-5.0 microns for films generally having a thickness of between about 1 to about 6 mils prior to stretching.

Examples of the inorganic fillers include calcium carbonate, talc, clay, kaolin, silica, diatomaceous earth, magnesium carbonate, barium carbonate, magnesium, sulfate, barium sulfate, calcium sulfate, aluminum hydroxide, zinc oxide, magnesium hydroxide, calcium oxide, magnesium oxide, titanium oxide, alumina, mica, glass powder, zeolite, silica clay, etc. Calcium carbonate (CaCO₃) is particularly preferred for its low cost, its whiteness, its inertness, and its availability. The selected inorganic filler such as calcium carbonate preferably is surface treated to be hydrophobic so that the filler can repel water to reduce agglomeration. Also, the surface treatment of the filler may improve binding of the filler to the thermoplastic polymer precursor while allowing the filler to be pulled away from the precursor film under stress. A preferred coating for the filler is calcium stearate, which is FDA compliant and readily available. Organic fillers such as wood powder, and other cellulose type powders also may be used. Polymer powders such as Teflon powder and Kevlar powder also can be used. The amount of filler added to the polyolefin precursor depends on the desired properties of the film. However, it is particularly preferred to produce films having good WVTR with an amount of filler greater than about twenty percent (20%) by weight of the thermoplastic resin/filler blend.

It is particularly preferred to use more than about twenty percent by weight of filler to assure the interconnection within the polymeric precursor film of voids created at the situs of the filler—particularly by the stretching operation to be subsequently performed. Further, it is particularly preferred to produce films with an amount of the filler less than about seventy percent (70%) by weight of the polymeric resin/filler composition. Amounts of filler greater than about 70% by weight may cause difficulty in compounding. Preferred ranges for the amount of fillers used in the present invention include from about 30% to about 70% by weight, more preferably from about 40% to about 60% by weight. Alternatively, no filler can be used to form the films of the invention.

While a broad range of fillers has been described at a broad range of inclusion parameters based on weight percentages, still other embodiments of the present invention are contemplated. For instance, fillers with much higher or much lower specific gravity may be included with the polymeric precursor at amounts outside the weight ranges disclosed. Such combinations will be understood to be contemplated as embodiments of our invention.

### Extrusion Process

The materials of the invention preferably are mixed and heated in a mixing and heating apparatus. Any mixing and heating apparatus and method can be used in the invention, and particularly preferred mixing and heating apparatus and methods are extrusion apparatus and processes. Extrusion processes are well known in the art, and any suitable extrusion process can be used to prepare the superabsorbent webs of the present invention, using the guidelines provided herein. These extrusion processes usually comprise mechanisms for feeding materials to the extruder, mechanisms for melting and mixing materials, mechanisms for transporting the molten materials to a forming die, and mechanisms for cooling the molten sheet of polymer to form a polymer film. In case a second film or web is laminated to the molten sheet, such second film or web may participate in the cooling process.

Methods and apparatus suitable for feeding the raw materials to the extruder generally are known. A preferred feeding mechanism comprises a conveying mechanism such as a vacuum pump connected to a vacuum pipe, the pipe being submerged in a reservoir of polymer material. In a controlled manner the pump generates vacuum in the pipe causing the pipe to suction polymer from the reservoir and to deposit it in a feed hopper. The feed hopper typically contains a metering device that deposits accurately controlled amounts of polymer into the extruder receiving cavity. Multiple cavities and feed hoppers may be present in a single extruder thereby enabling feeding of multiple components. In addition, antistatic and vibratory devices can be positioned at or near the feed hoppers to assist in accurately dosing the polymer. Other feeding mechanisms known to those skilled in the art or later discovered also are contemplated for use in the present invention.

A particularly preferred extruder for use in forming the superabsorbent web of the invention is a twin-screw extruder. Twin screw extruders of varying sizes are available from Thermo Haake, Hamburg, Germany. The receiving cavity(ies) may be positioned at various points along the length of the extruder barrel. The extruder screws rotate inside the barrel and thereby melt, mix, and transport various polymers and fillers received by various feed hoppers to the melt forming die. A preferred melt forming die is a cast die, but other types of dies are possible such as blown film dies. The die forms a molten polymer sheet that is subsequently cooled to create a film or a laminate structure.

In an alternative arrangement the molten polymer exits the extruder through a pelletizing die (a flat, cylindrical plate with multiple small openings). As the polymer passes through the die it forms strings of polymer. The strings may be subsequently cooled and cut by a rotating knife, and the cut strings are called “compounded pellets.” Compounded pellets then can be transported to a second extruder where they are melted again, transported to a die, and formed into a sheet that is subsequently cooled to form a film or laminate structure. In yet another alternative arrangement, the compounded pellets are combined with other polymer pellets in the second extruder.

Cooling mechanisms also are well known in the art, and any cooling mechanism now known or later discovered can be used in the present invention. A primary cooling mechanism can include an embossing station comprising two cooled rolls that are pressed against each other. The molten polymer is caused to pass between the embossing rolls (called engraving and anvil rolls, respectively) where it is cooled by contact with the cooler rolls. Alternatively, the rolls can both be smooth chill rolls without an engraving or embossing roll. Another well known cooling device comprises passing the polymer sheet over a single roll and applying an air or cool water curtain to the molten polymer to cause it to contact the single cooling roll. Both the air curtain and the contact with the roll contribute to cooling.

Another well known cooling mechanism comprises passing the polymer sheet over an apertured screen while in the presence of vacuum. Vacuum causes the polymer sheet to come into close contact with the screen causing the polymer to cool. In one embodiment the vacuum and screen combination cause the polymer sheet to conform to the shape of the apertured screen surface to form protrusions in the film. The side of the film that contacts the screen is called the formed film inner surface and the side of the film that is opposite the inner surface is called the formed film outer surface. The protrusions can be apertured, or they can be unapertured. Forming apertured polymer films in this manner is well known in the art.

### Lamination and Bonding

Commonly assigned U.S. Pat. Nos. 4,995,930, 5,591,510, 5,635,275, 5,635,276, 5,660,882, 5,698,054, 5,762,643, 5,733,628, 5,783,014, 6,242,074, and 6,303,208 describe a lamination technique called vacuum formed lamination (VFL), whereby a web substrate is laid upon a molten polymer sheet as successive portions of the sheet are passed over an apertured screen under the presence of vacuum. The disclosures of each of these patents is incorporated by reference herein in their entirety. The web substrate can be a nonwoven or it may be a thin polymeric substrate, breathable or non-breathable. The substrate may be a monolayer or multilayer substrate.

Other lamination techniques such as extrusion lamination, point bonding, ultrasonic bonding, and also adhesive bonding are contemplated as part of the invention.

### Stretching

Preparation of a stretched superabsorbent web can be achieved by stretching the precursor web to form interconnected voids. Stretching or “orientation” can be achieved by any number of methods known in the art such as for instance machine direction orientation, transverse direction orientation, intermeshing gear orientation (IMG) and others.

U.S. Pat. No. 6,264,864 and commonly assigned PCT Publication No.

WO99/22930 describe common precursor formulations and known stretching processes contemplated as part of the invention. The disclosures of these documents are incorporated by reference herein in their entirety.

In IMG stretching, a precursor web is incrementally oriented in the machine direction, transverse direction, or both. Films can be incrementally oriented by a number of mechanical techniques, however, the preferred technique is to stretch the film through pairs of intermeshing gears as shown in FIG. 11. Therein it may be seen that the film is contracted by the apex 18 of a plurality of teeth spaced a distance or pitch (W) apart. The apex 18 of each tooth extends into the open space 20 between the teeth on an opposing roller. The amount of engagement depends both on the tooth depth (d) and the relative position of the rollers.

IMG machine direction orientation typically is accomplished by stretching the film through a gear like pair of rollers 16 as shown in FIG. 12. IMG transverse direction orientation is accomplished by stretching the film through a pair of disk-like rollers as shown in FIG. 13.

The preferred embodiment employs rollers with a tooth pitch, W=0.066″, however a pitch of about 0.040″ to 0.150″ also is acceptable. The tooth depth (d), preferably is 0.100″, however, a tooth depth of about 0.030″ to 0.500″ also is acceptable. For the transverse direction orientation rollers, as shown in FIG. 13, the depth may be up to about 1.000″ as mechanical interference is less of an issue with the transverse direction rollers. A particularly preferred embodiment of the invention employs IMG rollers that can be temperature controlled from about 50° F. to about 210° F. More preferred is a temperature range of from about 70° F. to about 190° F. Even more preferred temperature ranges for use in the invention range anywhere from about 85° F. to about 180° F., and a most preferred temperature range is from about 95° F. to about 160° F. The roll temperature may be maintained through use of an internal flow of a heated or cooled liquid, an electrical system, an external source of cooling/heating, combinations thereof, and other temperature control and maintenance methods that will be apparent to those of ordinary skill in the art. The preferred embodiment is internal flow of a heated or cooled liquid through the rollers.

The depth of engagement of the roller teeth determines the amount of orientation imparted on the web. A balance usually is drawn between the depth of engagement of the roller teeth and the precursor web composition, as many physical properties of the web are affected. Some of the factors affecting the choice of pitch, teeth depth, and depth of engagement include the composition of the web, desired final properties (breathability, absorbency, strength, cloth-feel), and the width of the IMG rollers. The final application of the web also affects these choices because it determines desired final properties. The width of the IMG rollers presents economic and technical limitations—as the width increases the weight of the rollers also increases and so does the amount of deflection experienced by the rollers. Deflection creates variation not only in the process of stretching, but also in the process of making the rollers, particularly as the pitch and tooth depth increases.

Turning now to FIG. 1, a preferred process for the continuous creation of extruded superabsorbent web 58 preferably begins with the at least one thermoplastic resin 10 at room temperature, typically in pellet form. The at least one superabsorbent polymer 12, such as superabsorbent polymer powder, may be added to the resin 10 at a ratio of between about 5% to about 90% SAP by weight, and more preferably between about 30% to about 80% SAP by weight, even more preferably, from about 30 to about 70% by weight, more preferably from about 30 to about 65% by weight, more preferably from about 30 to about 55% by weight, and most preferably from about 45 to about 55% by weight. The resin and absorbent material may optionally be blended in dry mixer 20 (see, FIG. 1B) to create a dry blend 22, if desired, or the resin 10 and SAP 12 may be fed to the extruder 24 separately and mixed therein. It is preferred not to blend the resin 10 and superabsorbent polymer 12, when the polymer 12 is in the form of a powder. Blending can take place, however, when the superabsorbent polymer 12 is in the form of flakes or fibers.

The respective components can be fed into extruder 24, preferably, twin-screw extruder 24, where the resin 10 and SAP 12 then may be heated to melt the thermoplastic resin 10 and to form a molten blend 26. The molten blend 26 then can be transported by the extruder to an extrusion die 44 where it is formed into a molten sheet 46. The molten sheet 46 then may be cooled by a cooling device 52, e.g., by placing it contact with a cool surface 52 such as an embossing or chill roll to form a cooled sheet 54. The cooled sheet 54 can be utilized as is (an extruded absorbent web), or can be subsequently stretched in stretching apparatus 56 to form an extruded superabsorbent web 58. Stretching apparatus 56 can include any apparatus capable of stretching cooled sheet 54 in the machine direction, transverse direction, or both. Such apparatus include tenter frames, feed rollers rotating at variable speeds, stretching apparatus equipped with IMG, and the like. The extruded superabsorbent web 58 can be wound into a roll 60, if desired.

An alternative embodiment of the method of the invention is depicted in FIGS. 1A and 1B. In FIG. 1A, the molten sheet 46 can be cooled by cooling device 52 to form cooled sheet 54, which is utilized as is (i.e., without stretching) to form extruded web 58. In FIG. 1B, SAP 12 first is compounded in extruder 24 by mixing resin 10 and SAP 8 to form compounded SAP 12. Compounded SAP 12 then can be fed directly to a second extruder 24, optionally together with the same or different resin 14 to form the molten blend 26. Compounded SAP 12 may be dry blended with resin 14 in dry mixer 20 to create a dry blend 22, if desired. Dry blend 22 then can be fed to the second extruder 24 together with any optional additives and the like to form molten blend 26. Molten blend 26 then can be transported by the extruder to an extrusion die 44 where it is formed into a molten sheet 46. The molten sheet 46 then may be cooled by a cooling device 52, e.g., by placing it contact with a cool surface 52 such as an embossing roll to form a cooled sheet 54. The cooled sheet 54 can be subsequently stretched in stretching apparatus 56 to form an extruded superabsorbent web 58. The extruded superabsorbent web 58 then can be wound into a roll 60.

Turning now to FIG. 2, a preferred process for continuous creation of an extruded superabsorbent web 58 comprising at least two layer begins with the at least one thermoplastic resin 10 at room temperature, typically in pellet form. The at least one absorbent polymer 12, such as superabsorbent polymer powder, preferably is added to the resin 10 in an amount of between about 5% to about 90% SAP by weight, and more preferably between about 30% to about 80% SAP by weight, even more preferably, from about 30 to about 70% by weight, more preferably from about 30 to about 65% by weight, more preferably from about 30 to about 55% by weight, and most preferably from about 45 to about 55% by weight. The resin 10 and absorbent material 12 may optionally be blended in dry mixer 20 to create a dry blend 22, as described above with reference to FIG. 1B, although it is preferred not to blend the components when the superabsorbent material is in the form of a powder. The thermoplastic resin 10 and absorbent material 12 may be fed into an extruder 24 where the materials are heated to melt the thermoplastic resin 10 and to form a molten blend 26.

The molten blend 26 then can be transported by the extruder to an extrusion die 44 where it is formed into a molten sheet 46. The molten sheet 46 can be cooled by placing it contact with nonwoven web 50 and/or with a cooling device 52, e.g., cool surface 52 such as an embossing or chill roll. The molten web 46 and the nonwoven web 50 bond to form a cooled laminate 54. The cooled laminate 54 can be utilized as is (an extruded absorbent web), or can be subsequently stretched in a stretching apparatus 56 to form an extruded superabsorbent web 58. The extruded superabsorbent web 58 then can be wound into a roll 60. Those skilled in the art are capable of modifying the apparatus depicted in any one of FIGS. 1-3 to add additional layers to the superabsorbent web 58, using the guidelines provided herein.

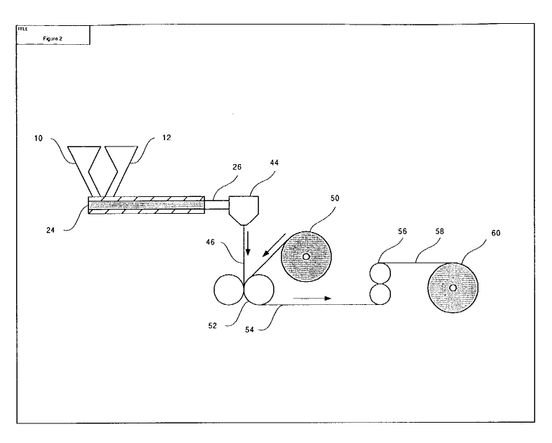

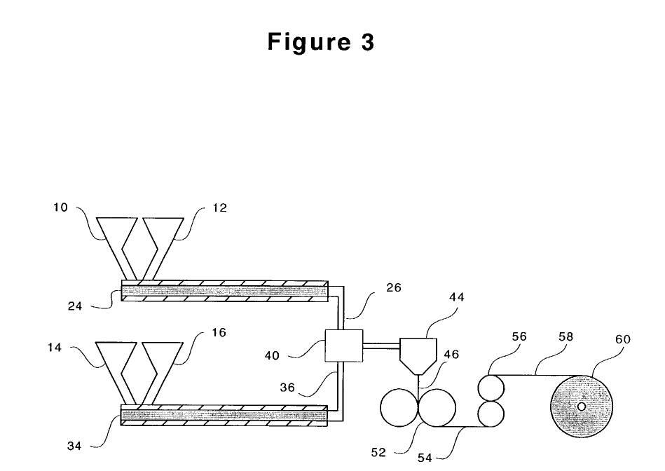

Turning now to FIG. 3, another preferred process for continuous creation of the extruded superabsorbent web 58 begins with the creation of a first layer comprising at least one thermoplastic resin 10 and at least one SAP polymer 12. The at least one thermoplastic resin 10 preferably is fed to the throat of an extruder, preferably a twin-screw extruder 24 at room temperature, typically in pellet form. The at least one superabsorbent polymer 12, such as SAP powder, also preferably is fed to the throat of, for example, a twin-screw extruder 24 at room temperature, typically in pellet form. The weight ratio of SAP to thermoplastic resin preferably is at a ratio of between about 5% to about 90% SAP to about 10% to about 95% resin, and more preferably between about 30% to about 80% SAP to about 20% to about 70% resin, even more preferably, from about 30 to about 70% to about 30% to about 70% resin, more preferably from about 30 to about 65% to about 35% to about 70% resin, more preferably from about 30 to about 55% by weight SAP to about 45% to about 70% resin, and most preferably from about 45 to about 55% SAP to about 45% to about 55% resin. Optionally, the thermoplastic resin and superabsorbent material may be blended in dry mixer 20 to create a dry blend 22 (FIG. 1B). The individual resin 10 and superabsorbent polymer 12 materials may then be fed into an extruder 24 where the respective materials are heated to melt the thermoplastic resin 10 to form molten blend 26. Another option is to feed one or both resin 10 and superabsorbent polymer 12 materials (and other optional additives, etc.) to the extruder 24 at a point other than its throat. Different combinations of extruders and screw types are well known in the art and are useful, and in some cases the polymers can be introduced at different entry points provided for by the extruder manufacturers. The molten blend 26 then preferably is transported by the extruder to an extrusion die block 40.

At or about the same time, a second layer comprising at least one thermoplastic resin 14 and, optionally, absorbent polymer 16, can be created. The thermoplastic resins 14 used to form the second layer may be the same or different as the thermoplastic resins 10 used to form the first layer. In addition, the absorbent polymer 16 used to form the second layer may be the same or different as the absorbent polymer 12 used to form the first layer. The thermoplastic resin 14 preferably fed to the throat of the extruder 34 at room temperature, typically in pellet form. The optional absorbent polymer 16, such as super absorbent polymer powder, preferably is added to the resin 14 in an amount of between about 5% to about 90% SAP by weight, and more preferably between about 30% to about 80% SAP by weight, even more preferably, from about 30 to about 70% by weight, more preferably from about 30 to about 65% by weight, more preferably from about 30 to about 55% by weight, and most preferably from about 45 to about 55% by weight. The resin and absorbent material may optionally be blended in dry mixer (not shown) to create a dry blend. The dry blend 32, or the respective thermoplastic resin 14 and optional absorbent polymer 16 may be fed into an extruder 34 where the respective materials are heated to melt the thermoplastic resin 14 and form molten blend 36. The molten blend 36 then preferably is transported by the extruder to the extrusion die block 40 where it is combined with molten blend 26.

The combined layers preferably exit the die block 40 and proceed through extrusion die 44 where the molten layers are formed into a molten sheet 46. In an alternative process, die block 40 is not used and the molten layers 26 and 36 move directly from extruders 24 and 34 to the extrusion die 44. The molten sheet 46 can be cooled by placing it contact with an optional nonwoven web (not shown—embodiment shown in FIG. 2) and/or with cooling device 52, e.g., a cool surface 52 such as an embossing or chill roll. The molten web 46 and the optional nonwoven web may bond to form a cooled laminate 54. The cooled laminate 54 then can be utilized as is (an extruded absorbent web), or can be subsequently stretched in a stretching apparatus 56 to form an extruded superabsorbent web 58. The extruded superabsorbent web 58 then can be wound into a roll 60.

The absorbent structures produced by these methods can be used for a large variety of applications. For example, hygiene applications such as feminine hygiene pads and liners, baby diapers, adult incontinence applications, disposable underwear and wound care and packaging applications such as meat trays and other absorbent packaging material. The absorbent structure is not limited to these uses and may obviously be used wherever absorbent material is needed.

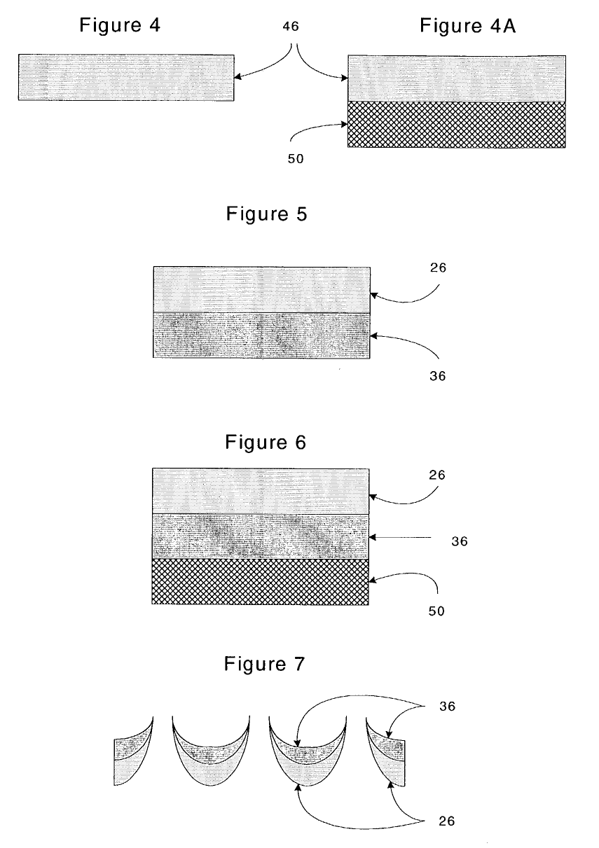

FIG. 4 shows a cross sectional view of an extruded superabsorbent web of this invention made according to the method described above with reference to FIG. 1. FIG. 4A shows a cross sectional view of an extruded superabsorbent web of this invention made according to the method described above with reference to FIG. 2. The first layer 46 is bonded to a nonwoven web 50 to form a composite material useful as a combination core/barrier laminate.

FIG. 5 shows a cross sectional view of an extruded superabsorbent web of this invention made according to the method described above with reference to FIG. 3 except that the method is modified to exclude the optional nonwoven layer. The first layer 26 is bonded to the second layer 36 to form a composite material useful as a combination core/barrier laminate. The second layer is primarily a breathable barrier layer although it may contain superabsorbent material to enhance its barrier properties. For example, the first layer 26 may be a SAP-containing layer, and second layer 36 may be a filler layer.

FIG. 6 shows a cross sectional view of an extruded superabsorbent web of this invention made according to the method described above with reference to FIG. 3. The first layer is the primary superabsorbent layer, the second layer is primarily a breathable barrier layer although it may contain superabsorbent material to enhance its barrier properties. The third layer is an optional nonwoven layer to provide cloth-like properties and feel.

FIG. 7 shows a cross sectional view of an extruded superabsorbent web of this invention made according to the method described above with reference to FIG. 3 except that the method is modified to exclude the optional nonwoven layer and also to exclude the stretching step. The first layer 26 is bonded to the second layer 36 to form a composite material. An alternative cooling apparatus 52 is used. Instead of using a solid cooling roll, an apertured roll is used, and vacuum is applied across the surface of the apertured roll. When the molten sheet 46 contacts the apertured roll it is caused by the vacuum to conform to the surface of the apertured roll, thereby forming protrusions. Such protrusions may be apertured or unapertured. An apertured web is shown in FIG. 7. This combination is useful as a breathable backsheet application whereby the male side of the film is placed facing the absorbent core. Molten sheet 36 is placed in contact with the apertured roll, and molten sheet 26 is located in contact with molten sheet 36 but on the opposite side from the side contacting the apertured roll. One of the primary purposes of the superabsorbent layer is to absorb any moisture that may pass through the male protrusion of the web.

FIG. 8 shows the web of FIG. 7 laminated to a nonwoven web 50.

FIG. 9 shows a cross sectional view of an extruded superabsorbent web of this invention made according to the method described above with reference to FIG. 3 except that the method is modified to exclude the optional nonwoven layer and also to exclude the stretching step. The first layer 26 is bonded to the second layer 36 to form a composite material. An alternative cooling apparatus 52 is used, whereby instead of using a solid cooling roll 52, an apertured roll is used. Vacuum is applied across the surface of the apertured roll. When the molten sheet 46 contacts the apertured roll it is caused by the vacuum to conform to the surface of the apertured roll, thereby forming protrusions. Such protrusions may be apertured or unapertured. An apertured web is shown in FIG. 9. Molten sheet 26 is placed in contact with the apertured roll, and molten sheet 36 is located in contact with molten sheet 26 but on the opposite side from the side contacting the apertured roll. This combination is useful as a topsheet or distribution layer application whereby the male side of the film is placed facing the absorbent core. One of the primary purposes of the superabsorbent layer is to assist in the fluid uptake function by providing additional absorption capacity.

FIG. 9A shows a formed film prepared by modifying the method illustrated in FIG. 1 by excluding the stretching step and replacing the cooling device with a vacuum forming apparatus as described immediately above with reference to FIG. 9. More specifically, instead of using a solid cooling roll 52, an apertured roll is used, and vacuum is applied across the surface of the apertured roll. When the molten sheet 46 contacts the apertured roll it is caused by the vacuum to conform to the surface of the apertured roll, thereby forming protrusions. Such protrusionsmay be apertured or unapertured. An apertured web is shown in FIG. 9A. FIG. 9B shows an apertured formed film made in accordance with the method used to make the formed film of FIG. 9A, with the exception that the formed film was stretched after being formed.

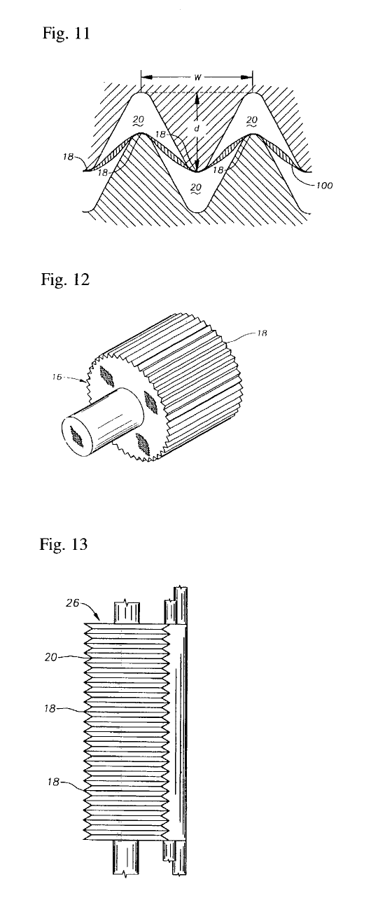

FIG. 10 shows a composite absorbent web designed to increase the absorption rate of the article by significantly increasing its capillary forces. The composite absorbent web is a layered arrangement of formed webs as shown in FIG. 9A. The layers can be laminated to one another after they have been vacuum formed. Once liquid penetrates the 2^{nd} or 3^{rd} layer of the composite web much more SAP is available quickly to absorb fluid. This construction is particularly attractive as a wipe product where the main objective is to absorb liquid and then to dispose of the wipe article.

The invention now will be described in greater detail with reference to the following testing procedures and examples.

### TEST METHODS

1. Absorbency

A test method was developed to measure the absorbency of various materials over time. The method is very similar to EDANA test method “FREE SWELL CAPACITY 440.1-99”. The method differs from the EDANA method in that a perforated film bag is used instead of a nonwoven tea bag so that material samples can be measured in addition to measuring free SAP. The method also differs in that the submersion and weighing process is repeated multiple times where the EDANA method is designed to weigh the tea bag only once. The method measures the amount of liquid absorbed by a substrate after the substrate is immersed in abundant quantity of liquid for a particular length of time. Water, saline solution, and synthetic blood are common liquids that can be used.

The test is also useful to measure free-swell capacity of SAP particles that are not bound to a structure, and is also useful to measure the absorption capacity of substrates where the SAP particles are mixed within a structure but not bound to any fiber or web.

### 1. Preparation of Testing Bag With Sample

A bag is made of a perforated plastic sheet with 11,500 holes per square inch. A preferred plastic sheet has three-dimensional protrusions exhibiting volcano shapes with apertures at both ends of each protrusion. These three-dimensional apertures have a maximum diameter at the base of the protrusion (where the protrusion originates in the sheet) of 150 microns (6 mils) and a smaller diameter at the far end of the protrusion (the end furthest away from the location where the protrusion originates in the sheet). The sheet allows liquid to enter the bag and to contact the sample, while also preventing particles from exiting the bag when the liquid is drained.

### 2. Preparation of Liquid Bed

A 5 cm (2 inch) deep pan of a size larger than the largest Testing Bag is filled with liquid (e.g., deionized water or saline solution).

### 3. Immersion of the Testing Bag

The Testing Bag is weighed, filled with a pre-determined weight of the sample, and then is immersed in the Liquid Bed and a stop-watch is started. Care is taken to ensure the Testing Bag is completely submerged in the liquid.

### 4. Measurement

After a predetermined length of time the Testing Bag is removed from the Liquid Bed and it is suspended to let liquid drain away for 2 minutes. After 2 minutes the Testing Bag has been observed to be substantially completely drained. The Testing Bag is weighed.

### 5. Measurement at Periodic Intervals

Steps 3-4 are repeated by re-submerging the Testing Bag in the Liquid Bed after weighing of the previous cycle is complete. For example, steps 3-4 can be repeated at 5, 15, 20, 60, and 120 minutes.

The difference in weight between the final weight of the Testing Bag and sample and the initial weight of the Testing Bag and sample represents the weight of fluid absorbed in the predetermined length of time.

### 2. Absorption Under Load

This method is used to evaluate the absorptive capacity of SAP-containing materials under the impetus of pressure. It follows the principle of EDANA 441.1-99, but has been adapted for use with absorptive cores.

A test portion is weighed and placed on a 400 mesh screen under a specified cylinder. A uniform pressure is applied to the cylinder/screen apparatus, which is placed in a container of a test solution. After a specified absorption time, the 400 mesh screen and cylinder are removed from the apparatus and weighed to determine the amount of solution absorbed.

This test can be performed on a SAP-containing material of a feminine napkin, panty liner or other hygiene products.

Reagents:

Test Solution: Analytical grade Saline 70 (0.9% saline) or Johnson, Moen Synthetic Blood Reagent with Red Dye, F-1670, Representative of human blood and other body fluid, not hazardous was used. Johnson, Moen and Co: 507-252-1766

Apparatus:

Analytical Balance: Accuracy 0.001 g

Paper towels

Timer: accurate to 0.1 seconds

Apparatus base: large enough to hold the AUL Apparatus and allow a liquid level of at least ½″ above the level of the screens.

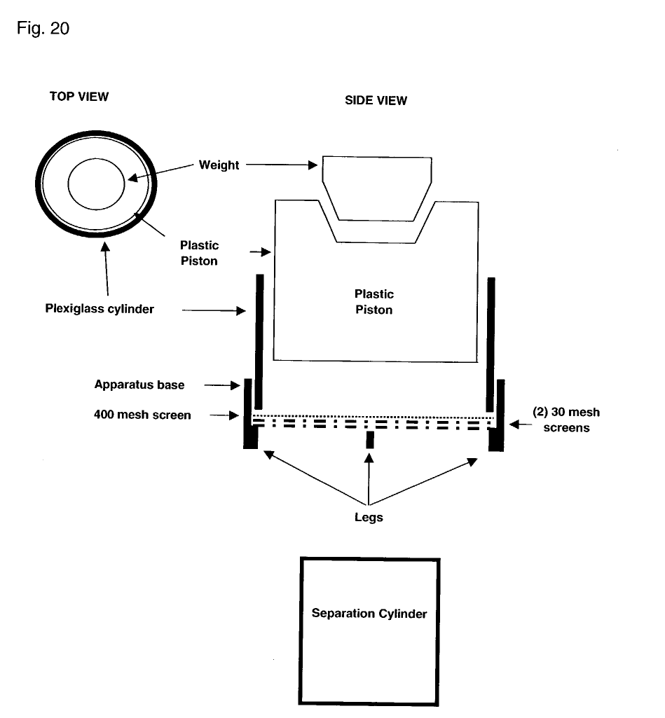

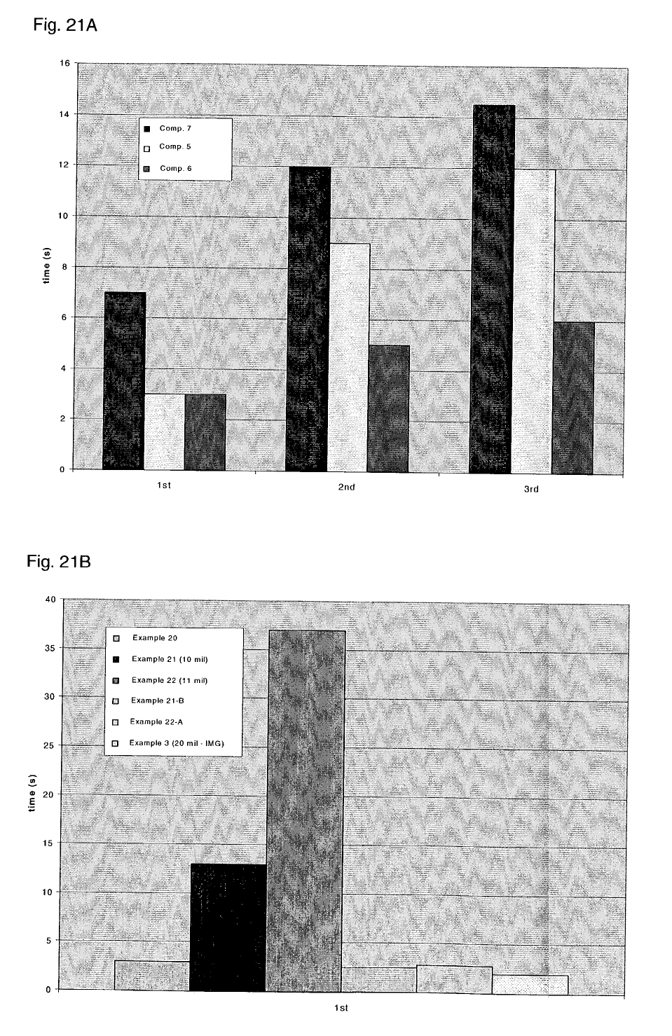

AUL Apparatus (see FIG. 20):

Plexiglas cylinder:

internal diameter: d1=(60±0.2) mm

height=(50±0.5) mm

400 (36 μm) mesh stainless steel screen

with (2) 30 mesh stainless screens under the 400 mesh

Plastic piston:

piston diameter d2 (mm) such that d1-d2=(0.8±0.2) mm

height (60±0.5) mm

Cylindrical weights (to be placed on top of Plastic piston):

| | | | Piston | |
|---|---|---|---|---|
| Piston | Load | Load | Area | Piston + |
| diameter (cm) | (psi) | (g/cm2) | (cm2) | Weight (g) |
| 5.92 | 0.3 | 21.09 | 27.52 | 580.6 |
| 5.92 | 0.5 | 35.15 | 27.52 | 967.6 |
| 5.92 | 0.6 | 42.18 | 27.52 | 1161.1 |
| 5.92 | 0.9 | 63.28 | 27.52 | 1741.7 |

Separation Cylinder:

Height was greater than the apparatus base height; and

Diameter was less than the apparatus base inner diameter.

Sample sizes smaller than 59.2 mm in width:

A smaller piston may be used with the same cylinder assembly as long as the dry sample is of the same diameter as the piston and the weights are adjusted to reflect the correct psi load.

Example, 49.2 mm sample width:

| Piston | Load | Load | Piston Area | Piston + |
|---|---|---|---|---|
| diameter (cm) | (psi) | (g/cm2) | (cm2) | Weight (g) |
| 4.92 | 0.3 | 21.09 | 19.01 | 401.0 |
| 4.92 | 0.5 | 35.15 | 19.01 | 668.3 |
| 4.92 | 0.6 | 42.18 | 19.01 | 802.0 |
| 4.92 | 0.9 | 63.28 | 19.01 | 1203.0 |

Sample Preparation:

Using the plastic piston as a template, the area to be tested was marked with a fine ballpoint pen;

The test area then was cut out with a pair of scissors, ensuring to stay inside the template line; and

The sample labeled.

Testing Times:

Testing times were (in minutes) 1, 2, 5, 15, 30, 60, 120 & 240. For each time step a dry sample was used. Testing was considered complete once the sample reached maximum capacity. Maximum capacity was indicated when the (W₃) Wet Apparatus/Sample Wt did not increase for three consecutive time intervals.

Sample Load:

Sample load was 0.5 psi unless a different load was noted.

Apparatus Assembly:

The two 30 mesh screens were placed into the apparatus base.

The 400 mesh screen was placed on top of the 30 mesh screens.

The plexiglass cylinder then was placed into the apparatus base, ensuring that it was tightly seated onto the screens.

Test Procedures:

1. Test Portion:

A sample was weighed dry and its weight recorded (W₁). Two dry samples were required for each time step.

2. Average Apparatus Tare (wet) Weight (W₂) was Measured as Follows:

a. The piston was placed in the cylinder.

b. The piston/cylinder was placed into the solution.

c. The timer was started once the solution appeared through the 400 mesh screen and the test was run for the specified time.

d. The piston/cylinder was removed from the solution.

e. The plexiglass cylinder was removed from the apparatus base by twisting and pulling upward.

f. The apparatus base was set on a separation cylinder so that the screen/piston combination was removed from the apparatus base.

g. The 2 bottom 30 mesh screens were slid out from the piston/screen combination.

h. The piston/400 mesh screen combination was placed onto a dry paper towel for 5 seconds.

i. The piston/400 mesh screen combination was slid across the paper towel to a dry spot and allowed to stand for an additional 5 seconds.

j. The piston/400 mesh screen combination was placed onto the scale and its weight recorded.

k. All parts of the apparatus were cleaned and dried using compressed air to dry the 400 and 30 mesh screens.

l. Steps a-i above were repeated one more time and the average of the two tare weights (W₂) was used for the AUL calculations (see calculations below).

3. Wet Apparatus/Sample Weight (W₃) was Measured as Follows:

a. A dry sample was placed onto the 400 mesh screen in the cylinder

b. The piston was placed on the sample, ensuring the sample was completely covered by the piston.

c. The selected weight was placed onto the piston.

d. The piston/cylinder was placed into the solution.

e. The timer was started once the solution appeared through the 400 mesh screen and the test was run for the specified time.

f. The piston/cylinder was removed from the solution.

g. The weight was removed from the piston.

h. The plexiglass cylinder was removed from the apparatus base by twisting and pulling upward.

i. The apparatus base was set on the separation cylinder so that the screen/sample/piston combination was removed from the apparatus base.

j. The 2 bottom 30 mesh screens were slid from the piston/sample/screen combination.

k. The piston/sample/400 mesh screen combination was placed onto a dry paper towel for 5 seconds.

l. The piston/sample/400 mesh screen combination was slid across the paper towel to a dry spot and let stand for an additional 5 seconds.

m. The piston/sample/400 mesh screen combination was placed onto the scale and the weight recorded, and the sample discarded.

n. All parts of the apparatus were cleaned and dried using compressed air to dry the 400 and 30 mesh screens.

Steps a-l were repeated one (1) more time to arrive at the two (2) Wet Apparatus/Sample Weights (W3) for the AUL calculations (see calculations below).

Calculations:

$$AUL ( g \text{/} g ) = \frac{( W3 ) ( g ) - ( W2 ) ( g ) - ( W1 ) ( g )}{( W1 ) ( g )}$$

AUL(g)=(W3)(g)−(W2)(g)−(W1)(g)

3. Multiple Strikethrough

This method is used to evaluate the multiple liquid acquisition and rewet performance of cores and samples of pantiliners and pads (i.e., core+acquisition distribution layer (ADL)+topsheet) using a strikethrough plate and saline solution. The procedure follows the general principles outlined in EDANA 150.4-99 and 151.2-99.

A sample of pad or pantiliner was placed on a Plexiglas base. The assembly was compressed with an 800 g strikethrough plate having 4×4 in. base dimension. A specific amount of test fluid was then dispensed from about 1 in height onto the surface of the topsheet through the star-shaped hole in the center of the strikethrough plate. A current was registered as the liquid connected the electrodes embedded around the hole and the clock started the timing. As the fluid penetrated through the topsheet, the electrodes were disconnected again and the current was cut off, and the clock stopped. The time elapsed was recorded as the first strikethrough time.

A standard weight was placed on the sample to ensure even spreading of the liquid. A pre-weighed pickup paper then was placed on the sample and the weight again was placed on the sample. The mass of liquid absorbed by the pick up paper was defined as first rewet.

The strikethrough and rewet test then were repeated 2 more times.

Apparatus:

Strikethrough plate: Refer to EDANA 151.2-99 for design. This procedure used a total weight of 805 g with 4×4 in base dimension.

Electronic Timer: measuring to nearest 0.01 s.

Stopwatch: measuring to nearest 0.1 s.

Base support: Transparent Plexiglas, 5×5 in and 5 mm thick.

Rewet Weight: Consisted of two parts: 1) Lexan disk with 45 mm diameter and 5 mm thickness, 11.1 grams, 2) Weight of 540±2 grams, with 45 mm diameter. Total weight for both was 550±2 grams. These two parts were attached.

Rewet Pickup paper: 5″×5″ paper #632, from EMC 512-948-1616

Electronic balance: capable of weighing to the nearest 0.0001 grams.

Liquid Dispensing Assembly:

Pipette: Variable Volume 0.5-5 ml, accuracy±0.1% VWR Scientific Cat#53499-605

Pipette Tips: for Variable Volume Pipette (cut to provide 3 mm diameter orifice), VWR Scientific Cat#53503-826

Separatory Funnel—125 ml

Burette—50 ml

Ring Stand and clamps—used to support the funnel and burette

Cutting tools: razor blades, scissors

Test Solution:

Saline 70—0.9% w/v NaCl solution, 70 dynes (mN/m)

Saline 45—0.9% w/v NaCl solution, w/surfactant, 45 dynes (mN/m), see QETM-00066 for solution preparation.

Instrument Preparation:

The 50 ml burette and Separator funnel were rinsed with the test solution at least two or three times prior to testing. One pipette tip was cut at an angle to provide a 3 mm diameter orifice at the end. The other end was trimmed to allow a snug fit on outlet of plastic separatory funnel.

The ring stand was set up with the separator funnel so that the tip of the funnel was 1-1/8″±1/32″ above the top of the Plexiglas base plate, 5″×5″.

The strikethrough plate was placed under the tip of the separator funnel, resting on the 5″×5″ Plexiglas base.

The power adapters from the timer unit were plugged in, and the power to the timer unit turned on.

The switch directly below the power switch was depressed to strikethrough. The apparatus was now ready for specimen evaluation.

Test Procedures:

Strikethrough:

The test sample (pad or pantiliner) to be evaluated was placed on the dry Plexiglas base plate, where the topsheet was exposed to the liquid.

The width and length of the pad or pantiliner were measured and recorded, making sure to prevent the pad from curling.

The dry strikethrough plate with the timer connector plugs in place was centered on the topsheet and the entire assembly was centered under the stem of the Separatory funnel.

The Separatory funnel was adjusted so that the tip of the funnel was close to the center of the strikethrough plate.

5 ml of the test solution was discharged for pantiliners (or 10 cc for pads, or specified by the engineer) from the Pipette or burette into the funnel.

The strikethrough test was started by suddenly opening the funnel stopcock and discharging the 5 ml (or 10 cc) of solution into the strikethrough plate cavity.

The initial solution discharge started the timer and after the solution had emptied from the cavity, the timer was automatically shut off.

After the timer had shut off, the time was recorded as first strikethrough time to the nearest 0.01 seconds and reset the timer.

Rewet:

The strikethrough plate was carefully removed without disturbing the test sample.

The dry rewet weight (the Lexan disk in contact with the sample) was gently and slowly placed on and centered over the test sample.

The timer (stopwatch) was started when the weight was in place, and ran for three minutes.

All 4 piles of the pick up paper then were weighed and the weight recorded.

After three minutes was shown by stopwatch, The rewet weight was removed

The pick up papers were centered on the sample.

The rewet weight was placed back on the pick up papers for two minutes.

After two minutes, the rewet weight was removed and the 4 piles of pick up paper were immediately weighed to the nearest 0.0001 gram and the weight recorded as the first rewet.

The rewet weight was dried, allowing no more than 1 minute to pass between removal of rewet weight and addition of strikethrough plate.

The strikethrough plate was placed back on the sample and steps 3-8 of the Strikethrough procedure, and steps 1-9 of the Rewet procedure were repeated two more times for the 2^{nd} and 3^{rd} Strikethrough and Rewet values.

The 2^{nd} and 3^{rd} strikethrough and rewet values were measured and recorded.

### EXAMPLES

This invention will be described further below with reference to working examples. It should be noted however, that this invention is not limited to these working examples.

### Examples 1-6

Three types of resins were used for these examples. The resins were mixed with two types of SAP particles to create 6 different combinations of resin and SAP. Typically the SAP had a 300 micron (6 mils) average particle size prior to use. The SAP was ground by passing it between a pair of rollers so that its average particle size was reduced to a 50 micron average particle size (2 mils). All 6 combinations were comprised of 65% thermoplastic resin by weight and 35% SAP.

Resin 1 was used in examples 1-2. Resin 1 was low density polyethylene 721 resin commercially available from The Dow Chemical Company, Midland Mich.

Resin 2 was used in examples 3-4. Resin 2 was ethylene vinyl acetate copolymer thermoplastic resin (EVA) containing 12% vinyl-acetate commercially available from E.I. DuPont de Nemours & Co., Wilmington, Del. under the trade name Elvax 3134Q.

Resin 3 was used in examples 5-6. Resin 3 was Optema TC-120, an ethylene methacrylic acid resin commercially available from Chevron Corporation, San Francisco, Calif.

SAP 1 was used in examples 1, 3 and 5. SAP 1 was SXM 880 commercially available from Stockhausen, Inc., Greensboro, N.C. The Free Swell Capacity of SXM 880 is 119 g water/g of SAP.

SAP 2 was used in examples 2, 4, 6, and 9. SAP 2 was FAVOR PAC 100 SAP polymer commercially available from Stockhausen Inc., Greensboro, N.C. The Free Swell Capacity of FAVOR PAC 100 was 176 g water/g SAP. The average particle size of the SAP 2 for examples 2, 4, and 6 was about 50 μm, and the average particle size for example 9 was about 15 μm.

### Examples 7, 8

Example 6 was repeated, except the amount of resin and SAP varied as shown in Table 1 below.

### Example 9

Example 6 was repeated, except that the amount of resin and SAP were varied as shown in Table 1 below, and the particle size of the SAP was 15 μm.

Superabsorbent webs were made from the above resin and SAP by feeding the materials to the throat of a twin-screw extruder. The polymers were mixed, melted, and transported by the extruder to a cast extrusion die where the molten polymers were formed into a sheet. The sheet was conveyed through and between a pair of cooling rolls to form an un-stretched extruded superabsorbent web. The 50 micron SAP and the un-stretched web were tested according to the Absorption Capacity test described above, and the results are shown in table 1 below. The PAC 100 SAP performed much better than the SXM 880 SAP. While not intending on being bound by any theory, it is believed that the differences in total absorption may be attributed to the differences in particle cross-linking and shape.

TABLE 1
Unstretched films

| | Example | | | | | | | | |
|---|---|---|---|---|---|---|---|---|---|
| Components | 1 | 2 | 3 | 4 | 5 | 6 | 7 | 8 | 9 |
| Resin 1 | 65 | 65 | | | | | | | |
| Resin 2 | | | 65 | 65 | | | 55 | 45 | 50 |
| Resin 3 | | | | | 65 | 65 | | | |
| SAP 1 | 35 | | 35 | | 35 | | | | |
| SAP 2 50 microns | | 35 | | 35 | | 35 | 45 | 55 | |
| SAP 2 15 microns | | | | | | | | | 50 |
| Thickness (microns) | 432 | 406 | 483 | 419 | 597 | 381 | 381 | 356 | — |
| Total Absorption(g water/g SAP) | 39 | 135 | 70 | 138 | 60 | 121 | 154 | 146 | — |

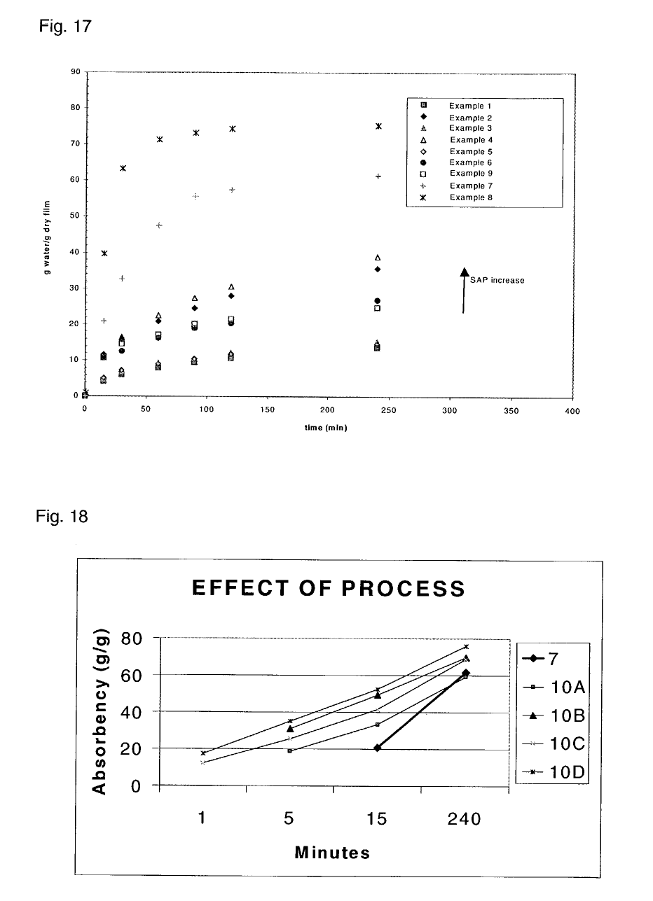

FIG. 17 illustrates the effect of SAP loading on absorbency. Examples 4, 7, and 8, which were otherwise identical except for the amount of SAP used (35, 45, 55 wt %, respectively), reveal that as the SAP loading increases, the total absorption generally increases. FIG. 17 also reveals that the particle size of the SAP may have an affect on absorbency. Example 9 contained 50% Resin 2 and 50% SAP 2 having an average particle size of 15 microns. The extruded web had a smooth surface, which indicates that SAP particles were not exposed at the surface, but were completely covered by the resin. In addition, the samples showed signs of agglomeration at some areas. While not intending on being bound by any theory of operation, it is believed that the agglomeration of the particles and lack of exposure on the film surface may have caused a decrease in the absorption capacity.

### Examples 10 and 11

Stretching Examples

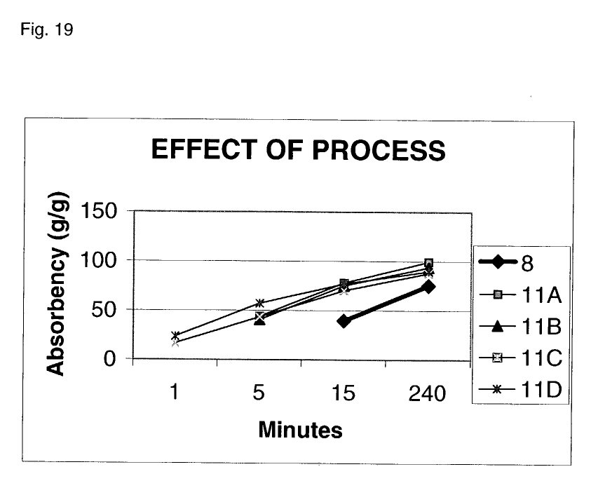

The process used in these examples was the same as that used to prepare examples 7 and 8 above. Only Resin 2 was used, and only SAP 2 having an average particle size of 100 microns was used at two levels: 45%, and 55%, for examples 7 and 8 respectively. The superabsorbent webs prepared in accordance with examples 7 and 8 were subjected to different stretching conditions as shown in Table 2 below. Stretching was carried out by subjecting the webs to IMG stretching and varying the pitch of the teeth of the gears, and by varying the degree of elongation (DOE), as shown in Table 2. The results and effect of stretching on absorbency are shown in FIGS. 18 and 19.

TABLE 2
Stretched films

| | | | Absorbency | | | | |
|---|---|---|---|---|---|---|---|
| | | | | | | | g liquid/ |
| | | | | | | | g SAP |
| | | | g liquid/g substrate | | | | Time |
| | | | Time (minutes | | | | (minutes) |
| Example | DOE | Pitch | 1 | 5 | 15 | 240 | 240 |
| 7 | 0 | 0 | | | 20.8 | 61.5 | 137.0 |
| 10A | 100 | 95 | | 18.6 | 33.3 | 59.3 | 132.0 |
| 10B | 150 | 95 | | 31.0 | 49.5 | 69.9 | 155.0 |
| 10C | 100 | 120 | 12.1 | 25.7 | 41.5 | 68.8 | 169.0 |
| 10D | 150 | 120 | 17.3 | 34.9 | 52.3 | 76.0 | 169.0 |
| 8 | 0 | 0 | | | 39.6 | 75.4 | 137.0 |
| 11A | 100 | 95 | | 44.1 | 78.2 | 99.5 | 181.0 |
| 11B | 120 | 95 | | 40.7 | 75.4 | 93.9 | 171.0 |
| 11C | 100 | 120 | 16.7 | 43.4 | 70.4 | 88.0 | 196.0 |
| 11D | 150 | 120 | 23.6 | 57.3 | 77.1 | 89.9 | 200.0 |

The results shown in table 2 above also are shown graphically in FIGS. 18 and 19. The results reveal that absorbency increases with the amount of SAP used, and surprisingly, that stretching increased the absorbency rate. Even more surprisingly the results show that the final absorbency of the stretched films exceeds the absorbency that would be expected had only SAP been used, even after only 240 minutes. For example, the free swell capacity of PAC 100 was 176 g/g (g liquid/g SAP) yet the absorbency of the stretched film of example 8, under stretching conditions D—sample 11D in Table 2 above, after only 240 minutes was 200 g/g. While not intending on being bound by any theory, the additional absorbency may be attributed to capillary and other effects resulting from the creation of voids during the stretching process. FIGS. 18 and 19 also reveal the effects various stretching conditions have on absorbency. These figures generally show that as stretching increases, absorbency increases, and that as the amount of SAP present in the film increases, the absorbency increases.

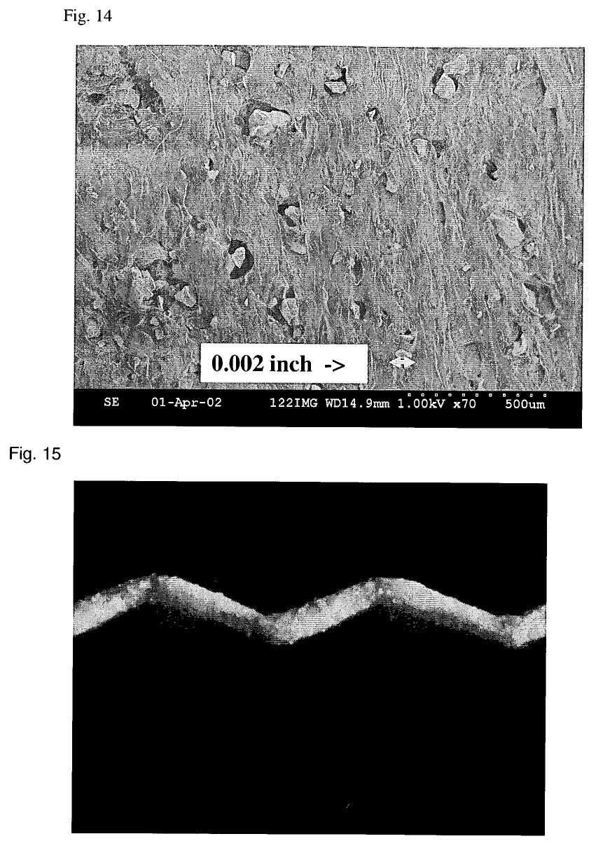

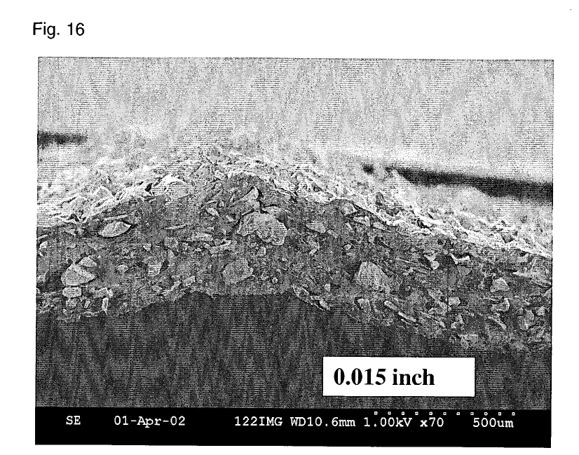

FIGS. 14-16 are photographs of the films of examples 11B (FIGS. 14 and 16) and 11A (FIG. 15). FIGS. 14 and 16 show the superabsorbent polymer particles dispersed within voids in the stretched films. FIG. 15 is a cross-sectional view of the stretched film of example 11A.

### Examples 12-20

Extruded superabsorbent webs were prepared in the same manner as described above with reference to Examples 1-9, except the resins and SAP were as shown in Tables 3 and 4 below.

TABLE 3
Raw Materials

| | | | Melting Point | Melt | |
|---|---|---|---|---|---|
| Resin ID | Manufacturer | Description | deg C. | Index | Density |
| Resin 1 (3134)* | DuPont | 12% VA EVA | 95 (203° F.) | 8 | 0.9360 |
| Resin 2 (3124) | DuPont | 9% VA EVA | 98 (208° F.) | 7 | 0.9360 |
| Resin 3 (2247A) | DOW | LLDPE | 123 (253° F.) | 2.3 | 0.9170 |
| Resin 4 (R4553) | Chevron | LDPE | | 4 | 0.9230 |
| Resin 5 (R2035) | DOW | LLDPE | 124 (255° F.) | 6 | 0.9190 |
| Resin 6 (PL1280) | DOW | ULDPE Affinity | | 6 | 0.9 |
| Resin 7 (Q100) | Basell | PP-EPR | 300-325 F. | 0.6 | 0.89 |
| SAP 2 | Stockhausen | SAP | Thermoset | NA | |
| Surfactant | Ampacet | Surfactant conc. (12 Ml | | 12 | 0.9200 |
| | | LDPE, 12% at | | | |
| | | 100) | | | |

*Trade designation.

TABLE 4

| | Example | | | | | | | |
|---|---|---|---|---|---|---|---|---|
| Components | 12 | 13 | 14 | 15 | 16 | 17 | 18 | 19 |
| Resin 1 | 55% | 47% | | | | | | |
| Resin 2 | | | 47% | | | | | |
| Resin 3 | | | | 47% | | | | |
| Resin 4 | | | | | 47% | | | |
| Resin 5 | | | | | | 47% | | |
| Resin 6 | | | | | | | | 47% |
| Resin 7 | | | | | | | 47% | |
| SAP 2 | 45% | 45% | 45% | 45% | 45% | 45% | 45% | 45% |
| Surfactant | | 8% | 8% | 8% | 8% | 8% | 8% | 8% |

Example 20 was prepared in the same manner as example 13, except prior to being cooled after extrusion, the web was passed over a rotating screen and subject to vacuum to form a vacuum formed film. Examples 12-20 then were subjected to the Absorption Capacity Test described above, and the results are shown in Table 5 below.

TABLE 5

| | | | | | | Basis |
|---|---|---|---|---|---|---|
| | | | | | Thickness | Weight |
| Example | Description | 1 min | 2 min | 30 min | (mil) | (gsm) |
| No Stretch | | | | | | |
| 12 | | 3 | 7 | 13 | 6 | 186 |
| 13-1 | smooth | 5 | 12 | 14 | 6 | 197 |
| 13-2 | rough | 5 | 8 | 14 | 18 | 270 |
| Smooth film stretch | | | | | | |
| 10D | 150 DOE | 4 | 6 | 10 | 10 | 202 |
| 12-A | 80 DOE | 6 | 10 | 13 | 6 | 148 |
| 13-A | 80 DOE | 7 | 12 | 15 | 5 | 151 |
| 14 | 70 DOE | 7 | 11 | 14 | 5 | 141 |
| 15 | 100 DOE | 7 | 11 | 13 | 5 | 121 |
| 16 | 40 DOE | 5 | 11 | 12 | 6 | 175 |
| 17 | 100 DOE | 7 | 12 | 13 | 5 | 108 |
| 19 | 120 DOE | 8 | 11 | 13 | 4 | 95 |
| Rough film stretch | | | | | | |
| 12-1-A | 70 DOE | 6 | 9 | 13 | 10 | 206 |
| 13-2-A | 70 DOE | 6 | 10 | 15 | 10 | 214 |
| 14 | 70 DOE | 7 | 10 | 14 | 9 | 181 |
| 15 | 70 DOE | 6 | 7 | 13 | 16 | 262 |
| 17 | 70 DOE | 5 | 9 | 13 | 14 | 193 |
| 19 | 70 DOE | 5 | 7 | 13 | 13 | 240 |
| 20 | no stretch | 6 | 8 | 9 | 31 | 125 |
| Comp. 1* | | 8 | 8 | 8 | 660 | 604 |
| Comp. 2 | | 9 | 8 | 8 | 84 | 158 |
| Comp. 3 | | 8 | 10 | 11 | 60 | 158 |
| Comp. 4 | | 13 | 18 | 26 | 42 | 212 |
| Comp. 5 | | 16 | 17 | 18 | 132 | 237 |
| Comp. 6 | | 7 | 7 | 7 | 79 | 134 |
| Comp. 7 | | 4 | 6 | 9 | 61 | 320 |

*all examples beginning with “comp.” denote comparative examples.

The Absorption Capacity data (g/g) show that addition of surfactant increased the absorption rate. Considering the ease of processing and stretchability, EVA, LDPE and plastomer resins are shown to be good candidates for absorbent cores. The data also indicate that for the inventive extruded webs, as the thickness decreased, the absorption rate (g/g) increased. This means that the basis weight of the sample also decreased, however, and thus the amount of liquid absorbed decreased. Thus, another method to compare the properties of conventional pads and pantiliners with the inventive superabsorbent webs was to compare their absorption capacity per m². Table 6 below provides this comparison.

TABLE 6

| | | | | |
|---|---|---|---|---|
| | Descrip- | Capacity/area | Capacity/area | Capacity/area |
| Example | tion | (1 min.) | (2 min) | (30 min) |

No Stretch

| | | | | |
|---|---|---|---|---|
| 12 | | 595 | 1375 | 2323 |
| 13-1 | smooth | 947 | 2268 | 2820 |
| 13-2 | rough | 1242 | 2187 | 3794 |

Smooth film stretch

| | | | | |
|---|---|---|---|---|
| 10D | 150 DOE | 808 | 1293 | 2041 |
| 12-A | 80 DOE | 834 | 1528 | 1909 |
| 13-A | 80 DOE | 988 | 1813 | 2261 |
| 14 | 70 DOE | 932 | 1609 | 1938 |
| 15 | 100 DOE | 843 | 1327 | 1559 |
| 16 | 40 DOE | 921 | 1848 | 2114 |
| 17 | 100 DOE | 798 | 1252 | 1381 |
| 19 | 120 DOE | 728 | 1077 | 1237 |

Rough film stretch

| | | | | |
|---|---|---|---|---|
| 12-1-A | 70 DOE | 1210 | 1830 | 2714 |
| 13-2-A | 70 DOE | 1327 | 2051 | 3168 |
| 14 | 70 DOE | 1218 | 1835 | 2478 |
| 15 | 70 DOE | 1448 | 1931 | 2240 |
| 17 | 70 DOE | 996 | 1672 | 2447 |
| 19 | 70 DOE | 1083 | 1799 | 3025 |
| 20 | no stretch | 786 | 973 | 1097 |
| Comp. 1 | | 5025 | 4959 | 4820 |
| Comp. 2 | | 1420 | 1336 | 1296 |
| Comp. 3 | | 1331 | 1569 | 1808 |
| Comp. 4 | | 2772 | 3809 | 5512 |
| Comp. 5 | | 3738 | 4049 | 4293 |
| Comp. 6 | | 899 | 911 | 966 |
| Comp. 7 | | 1368 | 1877 | 2855 |

The data show that the 1^{st} minute absorption for commercial pads is between 1300 to 2700 and for pantiliners is between 900 to 3700 g/m², respectively. The thinner sample (e.g., example 19, smooth stretch) has high g/g absorbency, but does not have enough g/m² absorbency to meet both pad and pantiliner requirements. It therefore was believed important to consider the combination of absorbency (based on g/g) and basis weight in assessing the overall absorbency of the inventive superabsorbent webs.

The vacuum formed film (example 20) had a similar absorption rate (i.e., 6 g/g) compared to other IMG stretched films at 1 minute, but had a lower total capacity (9 g/g). The data in Table 10 also show that there are various relationships between the absorption capacity and the basis weight of films. For example, there is a linear relationship between basis weight and capacity, e.g., as the film basis weight increases, the total absorbency per m² (absorption in 30 minutes) increases.

### Examples 21-23

To study the effect of thickness and % SAP on absorbency rate and capacity, and obtain the optimum thickness needed, a series of tests were performed using the Haake twin screw extruder used in examples 1-6. In these examples, SAP compounded films with thickness between 6-20 mils were made where % SAP were varied from 45-60%. In order to compare and screen cores, their absorbency under load and multiple strikethrough data were recorded. The raw materials used to prepare the extruded webs of examples 21-23 are summarized in Table 7 below.

TABLE 7

| Example | 21 | 22 | 23 |
|---|---|---|---|
| Resin 1 | 51% | 41.6% | 32.4% |
| SAP 2 | 45% | 55% | 65% |
| Surfactant | 4% | 3.4% | 2.6% |

Extruded superabsorbent webs of examples 21-23, were compared with conventional pantiliners (comp. 5-7) and pads (comp. 2-4) by measuring the absorbency under load of these samples using a load of 0.5 psi. The results are shown in Table 8 below.

TABLE 8

| | | | | | | | Basis |
|---|---|---|---|---|---|---|---|
| | | Thickness | | | | AUL | Weight |
| Example | Description | (mils) | AUL (1 min) | AUL (2 min) | AUL (5 min) | (30 min) | (gsm) |
| No Stretch | | | | | | | |
| 13-1¹ | smooth | 6 | 0.6 | 1.2 | 2.9 | 7.3 | 197 |
| 20 | rough | 31 | 4.0 | 4.7 | 5.5 | 6.7 | 125 |
| 21 | | 6 | 1.4 | 2.3 | 3.4 | 7.2 | 174 |
| Stretched webs | | | | | | | |
| 13-A² | 80 DOE | 6 | 4.6 | 6.0 | 7.2 | 8.9 | 145 |
| 21-A | 100 DOE | 6 | 5.8 | 6.9 | 7.9 | 11.0 | 112 |
| 21-B | 140 DOE | 10 | 4.9 | 5.4 | 6.8 | 9.9 | 147 |
| 21-C | 80 DOE | 6 | 2.7 | 3.5 | 4.5 | 8.1 | 144 |
| 21-D | 160 DOE | 15 | 3.7 | 4.6 | 5.1 | 8.4 | 201 |
| 22-A | 100 DOE | 11 | 4.8 | 5.8 | 7.4 | 11.2 | 205 |
| 22-B | 120 DOE | 15 | 3.4 | 3.7 | 5.0 | 9.0 | 237 |
| 23-A | 90 DOE | 9 | 3.6 | 4.4 | 5.9 | 9.1 | 191 |
| Comp. 2 | | 84 | 6.7 | 6.3 | 6.4 | 6.4 | 158 |
| Comp. 3 | | 60 | 6.3 | 6.8 | 7.1 | 8.3 | 158 |
| Comp. 4 | | 42 | 7.3 | 7.9 | 10.1 | 10.7 | 212 |
| Comp. 5 | | 132 | 12.9 | 13.1 | 15.4 | 14.3 | 237 |
| Comp. 6 | | 79 | 6.1 | 6.1 | 5.8 | 6.0 | 134 |
| Comp. 7 | | 61 | 3.4 | 4.0 | 4.7 | 6.0 | 320 |

¹Example 13 was repeated, except 45% SAP 2 was used, and surfactant was used in an amount of 1%.
²Example 13-1 was stretched at 80 DOE.

The samples were kept under load at various times and the saline solution was introduced to the samples from one side. Comparing the free absorption data in Tables 5 and 6 with the AUL data in Table 8 above reveal that, as would have been expected, the AUL values are lower than the free capacity values because of the load on the samples. For the superabsorbent webs of the invention, free absorption is about 40-80% higher than the AUL data. The difference is more pronounced for initial times. However, the AUL and free absorption data for commercially available materials (comp. 1-7) are less than 20-30% different. While not intending on being bound by any theory, it is believed that the reason is because the commercial materials are primarily non-woven and porous materials. Therefore, when one surface of the sample is exposed to the liquid, due to wicking action, liquid can travel relatively fast to the other side of the sample. Because the inventive samples are films with limited open area on their surface, they have limited capillary action and the liquid penetration mostly happens through diffusion process, which is a slower process. This is believed to be why the commercial samples reach their saturation level in about 15 minutes but most of the inventive samples needed more than 30 minutes to saturate.

### Example 24

This example compared the multiple strikethrough values for comparative examples 5-7, and inventive examples 20, 21 (10 mil), 22 (11 mil), 21-B, 22-A, and Example 3 (20 mil) that was subjected to IMG stretching. The top sheet material used for the inventive samples was a 25 mesh pentagonal arrayed vacuum formed film. The strikethrough values for the commercial materials are shown in FIG. 21A, and the strikethrough values for the inventive materials are shown in FIG. 21B.

As can be seen from the figures, the vacuum formed films (ex. 20) and the IMGed films (21-B, 22-A and 3) had strikethrough times similar to commercial cores, however, the second and third times could not be obtained for both IMGed and non-IMGed films. While not intending on being bound by any theory of operation, this was believed to be caused by the SAP swelling up and separating from the film after the first liquid acquisition, thereby blocking the liquid pathway. Therefore, liquid stayed on the stain area and did not distribute. On the other hand, the film made with 35% SAP 1 (i.e., SAP with low absorbency rate and capacity from Example 3) distributed the liquid well.

Based on the above results, effective distribution layers would be desirable to provide distribution across the materials to improve the 2^{nd} and 3^{rd} strikethrough times.

### Examples 25-32

To screen some ADLs, a set of multiple strikethrough tests were performed where different ADLs were evaluated when used with the same topsheet (a 25 mesh pentagonal arrayed vacuum formed film) and inventive material core (Example 22-A). The ADL for some of the examples below were inventive superabsorbent webs prepared in the previous examples. In addition, the ADL for some of the below examples contained more than one ADL layer.

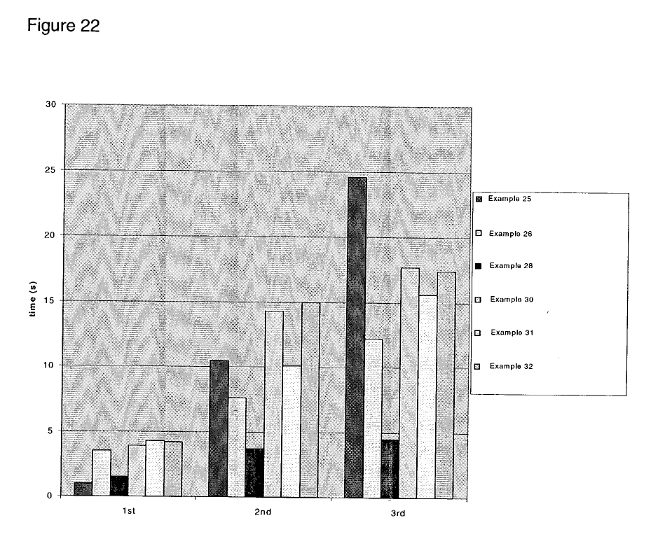

A summary of the data is shown in Table 9 and FIG. 22.

TABLE 9

| | | Strikethrough under | | |
|---|---|---|---|---|
| | | 0.3 psi | | |
| Example | ADL Description | 1st | 2nd | 3rd |
| 25 | ADL = AquaDri ™ (Tredegar) | 1 | 10.5 | 24.6 |
| 26 | ADL1 = VFF-Hex40, | 3.5 | 7.6 | 12.2 |
| | ADL2 = VFF-Hex60 | | | |
| 27 | ADL = Ex. 20 | 2 | — | — |
| 28 | ADL = Ex. 3 - stretched 160 DOE | 1.5 | 3.7 | 4.5 |
| 29 | ADL1 = VFF-Hex40, | 3.3 | 7.6 | 11.8 |
| | ADL2 = VFF-Hex60 | | | |
| | Core1 = Ex. 20 | | | |
| | Core2 = Ex. 22-A | | | |
| 30 | ADL = non-woven ADL from Kotex | 3.9 | 14.3 | 17.7 |
| | Ultra thin, gsm = 220, cellulose | | | |
| 31 | ADL = non-woven ADL from thin | 4.3 | 10.1 | 15.6 |
| | Ultra Always, gsm = 87, cellulose | | | |
| 32 | ADL = (coextruded layer of SAP | 4.2 | 15.0 | 17.4 |
| | and breathable backsheet, IMGed | | | |
| | 70 DOE, MD)* | | | |

*This ADL was manufactured by coextruding SAP and a backsheet formulation with three extruders, the first two containing the backsheet resins and the 3^{rd} containing the SAP 2. The backsheet formulation contained about 80% T928-E1 available from Ampacet, Terre Haute, In., 10% LD 202.48, and 10% 3518PA, both available from Exxon Chemical Company, Houston Texas.

VFF Hex 40 and VFF Hex 60 in Table 9 above are vacuum formed films having apertures in a hexagonal pattern with 40 mesh count and 60 mesh count, respectively. The term “mesh” as used herein to describe a regulated pattern means the square root of the maximum number of polygonal shapes (such as apertures) that can be inscribed in a flat square area measuring 1 inch (25,400 μ) on the side. For example, a tightly packed square pattern of apertures with 0.1 inch center-to-center distances is a 10 mesh square pattern. A tightly packed hexagonal pattern of apertures with 0.1 inch center-to-center distances is a 10.7 mesh hexagonal pattern (hexagonal patterns being packed more tightly than square patterns). A tightly packed hexagonal pattern of apertures with 0.01 inch center-to-center distances is a 107 mesh hexagonal pattern.

As is shown in FIG. 22, AquaDri™ (Tredegar Film Products, Richmond, Va.) improved the 1^{st} strikethrough time but the second and third strikethrough times still remained much higher than the commercial cores. When Hex-40 and Hex-60 layers were used as ADL1 and ADL2 (Example 26), they produced strikethrough times similar to commercial materials. In addition, these two layers provided the best liquid distribution among all the other ADLs tested (i.e., the core was wet completely after the 3 tests, and the 3^{rd} strikethrough times were still high). These two layers also helped the construction to feel soft and less film-like.

When the stretched film which contained 35% SXM 880 (Example 28) was used as an ADL, it showed comparatively low strikethrough times. SXM 880 is a SAP which has lower absorption rate and capacity which helps in distributing liquid without swelling up and blocking the liquid pathway. However, the acquisition times were too fast and since this film was too thick (20 mils) and did not have enough open area on its surface, it did not let the liquid to reach the core through the middle section and most of the liquid that was absorbed were through the two ends of the core. Thus, the liquid did not distribute well on the core and the middle section was dry. This layer could be effective if it were thinner and had more open area on its surface. Perforating this type of SAP film or coextruded films also could create effective ADLs.

The non-woven ADLs (Examples 30 and 31) not only distributed the liquid well but also created a reservoir for the liquid so that the liquid was initially absorbed by the ADLs and then slowly absorbed by the inventive materials. It also was shown that a planar capillary action existed between different IMGed layers (when intermeshed together). As long as the liquid was able to go through the first layer (high open area) and reach the second layer, it would be capable of distributing in the second core layer.

The invention has been described herein in reference to particularly preferred embodiments and examples. Those skilled in the art will appreciate, however, that various modifications may be made to the invention without departing from the spirit and scope thereof.

We claim:

1. An absorbent article comprising a wearer contacting surface and a garment contacting surface, a topsheet providing a wearer contacting surface, a backsheet providing a garment contacting surface, and an absorbent core disposed at least partially between the topsheet and the backsheet, at least one of the backsheet, the topsheet or the absorbent core comprising at least one layer of an extruded superabsorbent web, the extruded superabsorbent web comprising:
i. at least one thermoplastic polymer; and
ii. at least one superabsorbent polymer intermixed within the thermoplastic polymer.

2. The absorbent article of claim 1 where the thermoplastic polymer is present in an amount within the range of from about 20 to about 70% by weight, and the superabsorbent polymer is present in an amount within the range of from about 30 to about 80% by weight, based on the weight of the superabsorbent web.

3. The absorbent article of claim 1 where the thermoplastic polymer is present in an amount within the range of from about 30 to about 70% by weight and the superabsorbent polymer is present in an amount within the range of from about 30 to about 70% by weight, based on the weight of the superabsorbent web.

4. The absorbent article of claim 1 where the thermoplastic polymer is present in an amount within the range of from about 35 to about 70% by weight and the superabsorbent polymer is present in an amount within the range of from about 30 to about 65% by weight, based on the weight of the superabsorbent web.

5. The absorbent article of claim 1 where the thermoplastic polymer is present in an amount within the range of from about 45 to about 70% by weight and the superabsorbent polymer is present in an amount within the range of from about 30 to about 55% by weight, based on the weight of the superabsorbent web.

6. The absorbent article of claim 1 where the thermoplastic polymer is present in an amount within the range of from about 45 to about 55% by weight and the superabsorbent polymer is present in an amount within the range of from about 45 to about 55% by weight, based on the weight of the superabsorbent web.

7. The absorbent article of claim 1, wherein the superabsorbent web further comprises additives.

8. The absorbent article of claim 7, wherein the additives are fillers selected from the group consisting of calcium carbonate, talc, clay, kaolin, silica, diatomaceous earth, magnesium carbonate, barium carbonate, magnesium, sulfate, barium sulfate, calcium sulfate, aluminum hydroxide, zinc oxide, magnesium hydroxide, calcium oxide, magnesium oxide, titanium oxide, alumina, mica, glass powder, zeolite, and silica clay.

9. The absorbent article of claim 8, wherein the filler is calcium carbonate.

10. The absorbent article of claim 1, wherein the thermoplastic resin is selected from the group consisting of polyethylene, polypropylene, mixtures of polyethylene and polypropylene, ethylene polar co-monomer polymers, ethylene α-olefin copolymers, ethylene vinyl acetate (EVA), ethylene acrylic acid (EAA), ethylene methacrylic acid (EMA), polystyrene, polyesters, butadiene, elastomeric thermoplastic resins, and mixtures, combinations, and copolymers thereof.

11. An absorbent article comprising a wearer contacting surface and a garment contacting surface, a topsheet providing a wearer contacting surface, a backsheet providing a garment contacting surface, and an absorbent core disposed at least partially between the topsheet and the backsheet, at least one of the backsheet, the topsheet or the absorbent core comprising at least one layer including an extruded superabsorbent web, the extruded superabsorbent web comprising:
i. at least one thermoplastic polymer; and
ii. at least one superabsorbent polymer intermixed within the thermoplastic polymer; and
a second web bonded to the superabsorbent web.

12. The absorbent article of claim 11, where the second web is a polymeric web.

13. The absorbent article of claim 11, where the second web is a nonwoven web.

14. The absorbent article of claim 11, where the second web is a co-extruded film web.

15. The absorbent article of claim 14, where the co-extruded film web contains fillers.

16. The absorbent article of claim 15, where the fillers include superabsorbent polymers.

17. The absorbent article of claim 15, where the fillers include calcium carbonate.

18. The absorbent article of claim 11 where the thermoplastic polymer is present in an amount within the range of from about 20 to about 70% by weight and the superabsorbent polymer is present in an amount within the range of from about 30 to about 80% by weight, based on the weight of the superabsorbent web.

19. The absorbent article of claim 11 where the thermoplastic polymer is present in an amount within the range of from about 30 to about 70% by weight and the superabsorbent polymer is present in an amount within the range of from about 30 to about 70% by weight, based on the weight of the superabsorbent web.

20. The absorbent article of claim 11, where the thermoplastic polymer is present in an amount within the range of from about 35 to about 70% by weight and the superabsorbent polymer is present in an amount within the range of from about 30 to about 65% by weight, based on the weight of the superabsorbent web.

21. The absorbent article of claim 1, where the extruded superabsorbent web is stretched.

22. The absorbent article of claim 11, where at least the extruded superabsorbent web is stretched.

23. The absorbent article of claim 11, wherein the superabsorbent web further comprises additives.

24. The absorbent article of claim 23, wherein the additives are fillers selected from the group consisting of calcium carbonate, talc, clay, kaolin, silica, diatomaceous earth, magnesium carbonate, barium carbonate, magnesium, sulfate, barium sulfate, calcium sulfate, aluminum hydroxide, zinc oxide, magnesium hydroxide, calcium oxide, magnesium oxide, titanium oxide, alumina, mica, glass powder, zeolite, and silica clay.

25. The absorbent article of claim 24, wherein the filler is calcium carbonate.

26. The absorbent article of claim 11, wherein the thermoplastic resin is selected from the group consisting of polyethylene, polypropylene, mixtures of polyethylene and polypropylene, ethylene polar co-monomer polymers, ethylene α-olefin copolymers, ethylene vinyl acetate (EVA), ethylene acrylic acid (EAA), ethylene methacrylic acid (EMA), polystyrene, polyesters, butadiene, elastomeric thermoplastic resins, and mixtures, combinations, and copolymers thereof.

27. A superabsorbent web comprising:
i. at least one thermoplastic polymer; and
ii. at least one superabsorbent polymer intermixed within the thermoplastic polymer.

28. The superabsorbent web of claim 27, where the thermoplastic polymer is present in an amount within the range of from about 30 to about 70% by weight and the superabsorbent polymer is present in an amount within the range of from about 30 to about 70% by weight, based on the weight of the superabsorbent web.

29. The superabsorbent web of claim 27, where the thermoplastic polymer is present in an amount within the range of from about 45 to about 55% by weight and the superabsorbent polymer is present in an amount within the range of from about 45 to about 55% by weight, based on the weight of the superabsorbent web.

30. The superabsorbent web of claim 27, further comprising additives.

31. The superabsorbent web of claim 30, wherein the additives are fillers selected from the group consisting of calcium carbonate, talc, clay, kaolin, silica, diatomaceous earth, magnesium carbonate, barium carbonate, magnesium, sulfate, barium sulfate, calcium sulfate, aluminum hydroxide, zinc oxide, magnesium hydroxide, calcium oxide, magnesium oxide, titanium oxide, alumina, mica, glass powder, zeolite, and silica clay.

32. The superabsorbent web of claim 31, wherein the filler is calcium carbonate.

33. The superabsorbent web of claim 27, wherein the thermoplastic resin is selected from the group consisting of polyethylene, polypropylene, mixtures of polyethylene and polypropylene, ethylene polar co-monomer polymers, ethylene α-olefin copolymers, ethylene vinyl acetate (EVA), ethylene acrylic acid (EAA), ethylene methacrylic acid (EMA), polystyrene, polyesters, butadiene, elastomeric thermoplastic resins, and mixtures, combinations, and copolymers thereof.

34. A component useful in an absorbent article, the component comprising an extruded superabsorbent web comprising:
i. at least one thermoplastic polymer; and
ii. at least one superabsorbent polymer intermixed within the thermoplastic polymer; and
a second web bonded to the superabsorbent web.

35. The component of claim 34, where the second web is a polymeric web.

36. The component of claim 34, where the second web is a nonwoven web.

37. The component of claim 34, where the second web is a co-extruded film web.

38. The component of claim 37, where the co-extruded film web contains fillers.

39. The component of claim 38, where the fillers include superabsorbent polymers.

40. The component of claim 39, where the fillers include calcium carbonate.

41. The component of claim 34, where at least the extruded superabsorbent web is stretched.

42. The component of claim 34, where the thermoplastic polymer is present in an amount within the range of from about 45 to about 55% by weight and the superabsorbent polymer is present in an amount within the range of from about 45 to about 55% by weight, based on the weight of the superabsorbent web.

43. The component of claim 34, wherein the superabsorbent web further comprises additives.

44. The component of claim 43, wherein the additives are fillers selected from the group consisting of calcium carbonate, talc, clay, kaolin, silica, diatomaceous earth, magnesium carbonate, barium carbonate, magnesium, sulfate, barium sulfate, calcium sulfate, aluminum hydroxide, zinc oxide, magnesium hydroxide, calcium oxide, magnesium oxide, titanium oxide, alumina, mica, glass powder, zeolite, and silica clay.

45. The component of claim 44, wherein the filler is calcium carbonate.

46. The component of claim 34, wherein the thermoplastic resin is selected from the group consisting of polyethylene, polypropylene, mixtures of polyethylene and polypropylene, ethylene polar co-monomer polymers, ethylene α-olefin copolymers, ethylene vinyl acetate (EVA), ethylene acrylic acid (EAA), ethylene methacrylic acid (EMA), polystyrene, polyesters, butadiene, elastomeric thermoplastic resins, and mixtures, combinations, and copolymers thereof.

47. A method of making a superabsorbent web comprising:
providing a superabsorbent material and a polymeric resin to a mixing and heating apparatus;
heating and mixing the superabsorbent material and polymeric resin in the mixing and heating apparatus to melt the polymeric resin to form a molten blend of resin and superabsorbent material;
forming a molten sheet from the molten blend by passing the molten blend through a sheet forming apparatus; and
cooling the molten sheet to form a superabsorbent web.

48. The method of claim 47, further comprising stretching the superabsorbent web in the machine direction after cooling.

49. The method of claim 47, further comprising stretching the superabsorbent web in the transverse direction after cooling.

50. The method of claim 47, further comprising biaxially stretching the superabsorbent web in both the machine and transverse directions after cooling.

51. The method of claim 47, where the mixing and heating apparatus is an extruder.

52. The method of claim 51, where the extruder is a twin-screw extruder.

53. The method of claim 47, where cooling the molten sheet comprises passing the molten sheet over at least one cooling drum.

54. The method of claim 47, where cooling the molten sheet comprises passing a stream of cool air or water over the molten sheet.

55. The method of claim 47, where cooling the molten sheet comprises contacting the molten sheet with a second web.

56. The method of claim 55, where the second web is a polymeric web.

57. The method of claim 55, where the second web is a nonwoven web.

58. The method of claim 55, where the second web is a co-extruded film web.

59. The method of claim 58, where the co-extruded film web contains fillers.

60. The method of claim 47, further comprising compounding the superabsorbent polymer prior to providing the superabsorbent polymer to the heating and mixing apparatus, whereby compounding the superabsorbent polymer comprises heating and mixing the superabsorbent polymer with a polymeric resin to form a molten mixture of superabsorbent polymer and polymeric resin, and then pelletizing the molten mixture.

61. The method of claim 60, further comprising blending the polymeric resin and the compounded superabsorbent polymer in a blender to form a blended mixture, and then providing the blended mixture to the heating and mixing apparatus.

62. The method of claim 47, further comprising providing additives to the heating and mixing apparatus.

63. The method of claim 62, wherein the additives are fillers selected from the group consisting of calcium carbonate, talc, clay, kaolin, silica, diatomaceous earth, magnesium carbonate, barium carbonate, magnesium, sulfate, barium sulfate, calcium sulfate, aluminum hydroxide, zinc oxide, magnesium hydroxide, calcium oxide, magnesium oxide, titanium oxide, alumina, mica, glass powder, zeolite, and silica clay.

64. The method of claim 63, wherein the filler is calcium carbonate.

65. A superabsorbent web prepared by the method of claim 47.

66. A method of making an absorbent article comprising:
providing a topsheet comprising a wearer contacting surface, a backsheet comprising a garment contacting surface and an absorbent core to an article forming apparatus; and
disposing the absorbent core at least partially between the topsheet and the backsheet,
whereby at least the topsheet, backsheet, or absorbent core includes a superabsorbent web prepared by
providing a superabsorbent material and a polymeric resin to a mixing and heating apparatus;
heating and mixing the superabsorbent material and polymeric resin in the mixing and heating apparatus to melt the polymeric resin to form a molten blend of resin and superabsorbent material;
forming a molten sheet from the molten blend by passing the molten blend through a sheet forming apparatus; and
cooling the molten sheet to form a superabsorbent web.

67. The method of claim 66, further comprising stretching the superabsorbent web in the machine direction after cooling.

68. The method of claim 66, further comprising stretching the superabsorbent web in the transverse direction after cooling.

69. The method of claim 66, further comprising biaxially stretching the superabsorbent web in both the machine and transverse directions after cooling.

70. The method of claim 66, where the mixing and heating apparatus is an extruder.

71. The method of claim 70, where the extruder is a twin-screw extruder.

72. The method of claim 66, where cooling the molten sheet comprises passing the molten sheet over at least one cooling drum.

73. The method of claim 66, where cooling the molten sheet comprises passing a stream of cool air or water over the molten sheet.

74. The method of claim 66, where cooling the molten sheet comprises contacting the molten sheet with a second web.

75. The method of claim 74, where the second web is a polymeric web.

76. The method of claim 74, where the second web is a nonwoven web.

77. The method of claim 74, where the second web is a co-extruded film web.

78. The method of claim 77, where the co-extruded film web contains fillers.

79. The method of claim 78, where the fillers include superabsorbent polymers.

80. An absorbent article prepared by the method of claim 66.
